(19) United States
(12) Patent Application Publication
(10) Pub. No.: US 2023/0074785 A1
(43) Pub. Date: Mar. 9, 2023

(54) Method For Producing Recombinant Protein In Yeast Cells

(71) Applicant: BASF SE, Ludwigshafen am Rhein (DE)

(72) Inventors: Martin MAYHEW, San Diego, CA (US); Joseph Kerr GREDDER, San Diego, CA (US); Jacob LENIHAN, San Diego, CA (US); Paul HANDKE, San Diego, CA (US)

(21) Appl. No.: 17/792,659

(22) PCT Filed: Jan. 13, 2021
(86) PCT No.: PCT/US2021/013171

§ 371 Date: Jul. 13, 2022

Related U.S. Application Data
(60) Provisional application No. 62/960,831, filed on Jan. 14, 2020.

(51) Int. Cl. C12P 21/02; C12N 1/16; C12N 1/38

(52) U.S. Cl. CPC C12P 21/02; C12N 1/165; C12N 1/38; C12R 2001/84

(57) ABSTRACT

The present invention relates to a method for producing a recombinant protein in yeast cells, wherein the cells are subjected to a temperature shift at a specific timepoint of the cell culture. It also relates to a method for producing a recombinant protein in yeast cells by culturing said yeast cells in a medium having a high concentration of potassium ions compared to the concentration of sulfate and/or phosphate ions.

### FIELD OF THE INVENTION

The present invention relates to a method for producing a recombinant protein in yeast cells, wherein the cells are subjected to a temperature shift at a specific timepoint of the cell culture. It also relates to a method for producing a recombinant protein in yeast cells by culturing said yeast cells in a medium having a high concentration of potassium ions compared to the concentration of sulfate and/or phosphate ions.

### BACKGROUND OF THE INVENTION

The production of recombinant proteins in yeast cells is well-established. For example, Komagataella phaffii (formerly designated as Pichia pastoris) is a single-celled microorganism that is easy to manipulate and culture. K. phaffii is a eukaryote capable of many of the post-translational modifications performed by higher eukaryotic cells such as proteolytic processing, folding, disulfide bond formation and glycosylation. Thus, the K. phaffii system is preferred as an expression host cell over bacterial systems, which are not capable of performing the same post-translation modifications as eukaryotic cells. Further, in bacterial systems proteins may be lost, if they are produced in inactive inclusion bodies. The K. phaffii system has been shown to give higher expression levels of protein than many bacterial systems. Hence, foreign proteins requiring post-translational modifications may be produced as biologically active molecules in K. phaffii and K. phaffii is already used for the production of a wide variety of recombinant proteins.

WO 2014/145650 A1 discloses a method for protein production in yeast cells wherein the yeast cells expressing the protein are first cultured at a first temperature and then at a second different temperature.

Nevertheless, there is a need for a method which allows to produce recombinant proteins in yeast cells with a high yield.

### SUMMARY OF THE INVENTION

The present inventors have found cell culture parameters which lead to a particularly high titer of recombinant protein expressed by yeast cells. Furthermore, these cell culture parameters enhance the scalability of the cell culture process.

Accordingly, the present invention relates to a method for producing a recombinant protein in yeast cells, comprising the steps of:

(a) culturing yeast cells genetically modified to express the recombinant protein in a suitable cell culture medium comprising a first carbon source at a first temperature, thereby obtaining a first culture;
(b) feeding a second carbon source to said first culture when the first carbon source is depleted, wherein the first temperature is changed to a second temperature at the time the feeding of step (b) is initiated; and
(c) obtaining the recombinant protein.

In one embodiment the second temperature is lower than the first temperature.

In one embodiment the second temperature is 1 to 8° C. lower than the first temperature.

In one embodiment the first temperature is 30° C. In one embodiment the second temperature is 26° C.

In one embodiment first temperature is changed to the second temperature linearly over a period of time. In one embodiment the first temperature is changed to the second temperature linearly over a period of 30 minutes to two hours.

In one embodiment the culture pH is changed from a first pH to a second pH at the time the feeding of step (b) is initiated.

In one embodiment the second pH is higher than the first pH.

In one embodiment the second pH is 0.5 to 2 pH units higher than the first pH.

In one embodiment the first pH is 5.0. In one embodiment the second pH is 6.0.

In one embodiment the first pH is changed to the second pH linearly over a period of time. In one embodiment the first pH is changed to the second pH linearly over a period of 30 minutes to two hours.

In one embodiment the first and the second carbon source are the same.

In one embodiment the first and/or the second carbon source is glucose.

In one embodiment the culture medium is a defined medium.

In one embodiment the concentration of potassium ions in the culture medium is higher than the concentration of sulfate ions and/or phosphate ions.

In one embodiment the molar ratio of potassium ions to phosphate ions is 1.5:1 to 2.8:1.

In one embodiment the molar ratio of potassium ions to sulfate ions is 2:1 to 4:1.

In one embodiment the ammonium concentration in the culture medium is 5 mM to 80 mM.

In one embodiment the calcium concentration in the culture medium is 0.5 mM to 3 mM.

In one embodiment the magnesium concentration in the culture medium is 5 mM to 30 mM.

In one embodiment the feeding of the second carbon source is continuous.

In one embodiment the feed rate is increased exponentially from an initial feed rate to a maximum feed rate.

In one embodiment the feed rate is decreased linearly from the maximum feed rate to an intermediate feed rate and then decreased to a final feed rate.

The present invention also relates to a method for producing a recombinant protein in yeast cells, comprising the steps of:

(a) culturing yeast cells genetically modified to express the recombinant protein in a suitable cell culture medium comprising a first carbon source, potassium ions, sulfate ions and phosphate ions at a first temperature, wherein the concentration of the potassium ions in the culture medium is higher than the concentration of the sulfate ions and/or of the phosphate ions; and
(b) obtaining the recombinant protein.

In one embodiment the molar ratio of potassium ions to phosphate ions is 1.5:1 to 2.8:1.

In one embodiment the molar ratio of potassium ions to sulfate ions is 2:1 to 4:1. In one embodiment the ammonium concentration in the culture medium is 5 mM to 80 mM.

In one embodiment the calcium concentration in the culture medium is 0.5 mM to 3 mM.

In one embodiment the magnesium concentration in the culture medium is 5 mM to 30 mM.

In one embodiment the culture medium is a defined medium.

In on embodiment the method further comprises a step (a′) of feeding a second carbon source to the yeast cell culture of (a) when the first carbon source is depleted.

In one embodiment the first temperature is changed to a second temperature at the time the feeding of step (a′) is initiated.

In one embodiment the second temperature is lower than the first temperature.

In one embodiment the second temperature is 1 to 5° C. lower than the first temperature.

In one embodiment the first temperature is 30° C. In one embodiment the second temperature is 26° C.

In one embodiment the first temperature is changed to the second temperature linearly over a period of time.

In one embodiment the first temperature is changed to the second temperature linearly over a period of 30 minutes to two hours.

In one embodiment the culture pH is changed from a first pH to a second pH at the time the feeding of step (a′) is initiated.

In one embodiment the second pH is higher than the first pH.

In one embodiment the second pH is 0.5 to 2 pH units higher than the first pH.

In one embodiment the first pH is 5.0. In one embodiment the second pH is 6.0.

In one embodiment the first pH is changed to the second pH linearly over a period of time.

In one embodiment the first pH is changed to the second pH linearly over a period of 30 minutes to two hours.

In one embodiment the first and the second carbon source are the same.

In one embodiment the first and/or the second carbon source is glucose.

In one embodiment the feeding of the second carbon source is continuous.

In one embodiment the feed rate is increased exponentially from an initial feed rate to a maximum feed rate.

In one embodiment the feed rate is decreased linearly from the maximum feed rate to an intermediate feed rate and then decreased to a final feed rate.

In one embodiment the yeast is a methylotropic yeast. In one embodiment the yeast is Pichia pastoris.

In one embodiment the recombinant protein is expressed under the control of a promoter which is not inducible by methanol. In one embodiment the recombinant protein is expressed under the control of a promoter which is inducible by methanol.

In one embodiment the recombinant protein is an enzyme, a peptide, an antibody or antigen-binding fragment thereof, a protein antibiotic, a fusion protein, a vaccine or a vaccine-like protein or particle, a growth factor, a hormone or a cytokine.

In one embodiment the recombinant protein is a lipase, protease, alpha-amylase, beta-amylase, glucoamylase, xylanase, mannanase, glucanase, cellulase, or phytase.

### BRIEF DESCRIPTION OF THE DRAWINGS

FIG. 1: Product titer depending on the temperature in the production phase

FIG. 2: Product titer depending on culture media formulation

FIG. 3: Influence of glucose feeding strategy on the predictability between lab and pilot scale Dashed line: R&D scale; solid line: pilot scale

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given. Unless stated otherwise or apparent from the nature of the definition, the definitions apply to all methods and uses described herein.

As used in this specification and in the appended claims, the singular forms of “a” and “an” also include the respective plurals unless the context clearly dictates otherwise. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%.

It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only.

Furthermore, the terms “first”, “second”, “third” or “(a)”, “(b)”, “(c)”, “(d)” etc. and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay, there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

The term “yeast cell” has its typical meaning. Suitable yeast cells may be selected from the genus group consisting of Pichia, Candida, Torulopsis, Arxula, Hansenula, Ogatea, Yarrowia, Kluyveromyces, Saccharomyces and Komagataella. Preferably the yeast cell is from the genus Komagataella. More preferably, the yeast cell is Komagataella phaffii Within the present application the terms “Komagataella phaffii” and “Pichia pastoris” are used interchangeably.

In one embodiment, the yeast cell is a methylotrophic yeast cell. The term “methylotrophic yeast” as used herein includes, but is not limited to, for example, yeast species that can use reduced one-carbon compounds such as methanol or methane, and multi-carbon compounds that contain no carbon bonds, such as dimethyl ether and dimethylamine. For example, the methylotrophic yeast can use methanol as the sole carbon and energy source for cell growth. Without being limiting, methylotrophic yeast species may include the genus Methanoscacina, Methylococcus capsulatus, Hansenula polymorpha, Candida boidinii, Komagataella pastoris and Komagataella phaffii, for example. Preferably, the host cell is a Komagataella phaffii cell.

The term “genetically modified yeast cell” as used herein means that the yeast cell contains at least one nucleic acid sequence which is not naturally present in the cell or which is naturally present in the yeast cell, but linked to sequences to which it is not naturally linked in the yeast cell such as a promoter to which the nucleic acid sequence encoding a protein is not naturally linked.

The yeast cell is genetically modified by transforming it with at least one expression vector encoding the recombinant protein, i.e. an expression vector has been introduced into the yeast cell by the process of transformation. The presence of the an expression vector in the genetically modified yeast cell can be detected by methods such as PCR or Southern Blot which detect the nucleic acid sequence introduced into the yeast cell. Additionally or alternatively, the presence of the expression vector in the genetically modified yeast cell can be detected by detecting the expression of the recombinant protein by methods such as Western Blot or immunofluorescence.

In one embodiment, the yeast cell which is subjected to genetic modification is deficient for at least one auxotrophic marker and the expression vector may comprise a nucleic acid sequence encoding said auxotrophic marker. The recombinant yeast cell may be selected using said auxotrophic marker.

Suitable methods for transforming yeast cells and in particular Komagataella phaffii cells are known to the skilled person and are described for example in Pichia Protocols, 2nd edition 2007, edited by James M. Cregg, ISBN: 978-1-58829-429-6.

The expression vector comprises not only the expression cassette necessary for the expression of the recombinant protein, but also further elements enabling its propagation and selection in bacterial cells, such as an origin of replication functional in bacterial cells and an antibiotic resistance gene functional in bacteria which enables the selection of transformed bacteria.

The terms “nucleic acid”, “nucleic acid sequence” or “nucleic acid molecule” have their usual meaning and may include, but are not limited to, for example, polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. Nucleic acids can be either single-stranded or double-stranded. The term “isolated nucleic acid molecule” refers to a nucleic acid molecule that has been separated from the environment with which it is naturally associated, such as the genome.

The nucleic acid sequences used in the present invention further encompass codon-optimized sequences. A nucleic acid is codon-optimized by systematically altering codons in recombinant DNA to be expressed in a host cell other than the cell from which the nucleic acid was isolated so that the codons match the pattern of codon usage in the organism used for expression. Thereby the yields of an expressed protein are increased. The codon-optimized sequence nevertheless encodes a protein with the same amino acid sequence as the native protein.

The terms “coding for” or “encoding” as used herein have their usual meaning and may include, but are not limited to, for example, the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

The term “expression cassette” refers to a nucleic acid molecule containing the coding sequence of a protein and control sequences such as e.g. a promoter in operable linkage, so that host cells transformed or transfected with these sequences are capable of producing the encoded proteins.

The term “vector” refers to DNA sequences that are required for the transcription of cloned recombinant nucleotide sequences, i.e. of recombinant genes and the translation of their mRNA in a suitable host organism. Expression vectors comprise the expression cassette and additionally usually comprise an origin for autonomous replication in the host cells or a genome integration site, one or more selectable markers (e.g. an amino acid synthesis gene or a gene conferring resistance to antibiotics such as zeocin, kanamycin, G418 or hygromycin), a number of restriction enzyme cleavage sites, a suitable promoter sequence and a transcription terminator, which components are operably linked together.

The term “vector” as used herein includes autonomously replicating nucleotide sequences as well as genome integrating nucleotide sequences. Vectors include, but are not limited to, plasmids, minicircles, yeast integrative plasmids, episomal plasmids, centromere plasmids, artificial chromosomes and viral genomes. Available commercial vectors are known to those of skill in the art. Commercial vectors are available from European Molecular Biology Laboratory and Atum, for example.

In a preferred embodiment, the expression vector is a plasmid suitable for integration into the genome of the host cell, in a single copy or in multiple copies per cell. The nucleic acid sequence comprising the promoter and the nucleic acid sequence encoding the recombinant protein which is expressed under the control of the promoter may also be provided on an autonomously replicating plasmid in a single copy or in multiple copies per cell. The preferred plasmid is a eukaryotic expression vector, preferably a yeast expression vector. The expression vector may be any vector which is capable of replicating in or integrating into the genome of the host organisms. Preferably, the vector is functional in yeast cells such as Komagataella phaffii cells.

The vector can be produced by any method known in the art. For example, procedures to ligate the nucleic acid sequences encoding a protein and to insert the ligated sequences into a suitable vector are known and described for example in Green and Sambrook (2012) Molecular Cloning, 4th edition, Cold Spring Harbor Laboratory Press.

The term “promoter” as used herein refers to a nucleotide sequence that directs the transcription of a structural gene. In some embodiments, a promoter is located in the 5′ non-coding region of a gene, proximal to the transcriptional start site of a structural gene. Sequence elements within promoters that function in the initiation of transcription may also be characterized by consensus nucleotide sequences. These promoter elements include RNA polymerase binding sites, TATA sequences, CAAT sequences, differentiation-specific elements (DSEs; McGehee et al., Mol. Endocrinol. 7:551 (1993)), cyclic AMP response elements (CREs), serum response elements (SREs; Treisman, Seminars in Cancer Biol. 1:47 (1990)), glucocorticoid response elements (GREs), and binding sites for other transcription factors, such as CRE/ATF (O'Reilly et al., J. Biol. Chem. 267:19938 (1992)), AP2 (Ye et al., J. Biol. Chem. 269:25728 (1994)), SP1, cAMP response element binding protein (CREB; Loeken, Gene Expr. 3:253 (1993)) and octamer factors (see, in general, Watson et al., eds., Molecular Biology of the Gene, 4th ed. (The Benjamin/Cummings Publishing Company, Inc. 1987), and Lemaigre and Rousseau, Biochem. J. 303:1 (1994)). The promoter used in the context of the present invention may be inducible by methanol, i.e. the expression of the recombinant protein is induced by incubating the yeast cells with a suitable amount of methanol. One suitable methanol-inducible promoter is the AOX promoter according to SEQ ID NO: 5.

In another embodiment, a promoter is used which is not inducible by methanol. Such promoters include any of the promoters shown in SEQ ID NOs. 6, 7 and 8. Further promoters which are not inducible by methanol include the constitutive GAP promoter (Varnai et al. (2014) Microbial Cell Factories 13:57).

Further promoters for use in the present invention include those shown in SEQ ID NOs. 9, 10, 11 and 12.

The recombinant protein can be any protein such as any eukaryotic, prokaryotic and synthetic protein. The protein may be homologous to the host cell, i.e. it may be naturally expressed by the host cell, or it can be heterologous to the host cell, i.e. it may not be naturally expressed by the host cell. Preferably, the protein is heterologous to the host cell. The protein can include, but is not limited to, an enzyme, a peptide, an antibody or antigen-binding fragment thereof, a protein antibiotic, a fusion protein, a vaccine or a vaccine-like protein or particle, a growth factor, a hormone or a cytokine. Proteins obtained by heterologous expression in K. phaffi which are already on the market include phytase, trypsin, nitrate reductase, phospholipase C, collagen, proteinase K, ecallantide, ocriplasmin, human insulin, pleactasin peptide derivative NZ2114, elastase inhibitor, recombinant cytokines and growth factors, human cystatin C, HB-EGF, interferon-alpha 2b, human serum albumin and human angiostatin.

In one embodiment the recombinant protein is an enzyme. The enzyme may be selected from the group consisting of lipase, alpha-amylase, beta-amylase, glucoamylase, protease, xylanase, glucanase, cellulase, mannanase and phytase.

In one embodiment, the recombinant protein is a lipase. The lipase may have an amino acid sequence having at least 80% sequence identity to the amino acid sequence of SEQ ID No. 1. In one embodiment, the lipase has an amino acid sequence according to SEQ ID No. 1. In one embodiment, the lipase is encoded by a nucleic acid sequence having at least 80% sequence identity to the nucleic acid sequence of SEQ ID No. 2. In one embodiment, the lipase is encoded by the nucleic acid sequence according to SEQ ID No. 2. The protein having an amino acid sequence which is at least 80% identical to the amino acid sequence of SEQ ID No. 1 or which is encoded by a nucleic acid sequence which is at least 80% identical to the nucleic acid sequence of SEQ ID No. 2 has lipase activity.

The term “lipase activity” means that the protein can cleave ester bonds in lipids. The lipase activity of a protein can be determined by incubating the protein with a suitable lipase substrate, such as PNP-octanoate, 1-olein, galactolipids, phosphatidylcholine and triacylglycerols and determining the lipase activity in comparison to a control lipase.

In one embodiment, the lipase comprises one or more amino acid insertions, deletions or substitutions in comparison to the amino acid sequence of SEQ ID No. 1. In one embodiment, the amino acid insertion, deletion or substitution in comparison to the amino acid sequence of SEQ ID No. 1 is at an amino acid residue selected from amino acid residues 23, 33, 82, 83, 84, 85, 160, 199, 254, 255, 256, 258, 263, 264, 265, 268, 308 and 311. In one embodiment, the amino acid substitution in comparison to the amino acid sequence of SEQ ID No. 1 is selected from the group consisting of: Y23A, K33N, S82T, S83D, S83H, S831, S83N, S83R, S83T, S83Y, S84S, S84N, I85A, I85C, I85F, I85H, I85L, I85M, I85P, I85S, I85T, I85V, I85Y, K160N, P199I, P199V, I254A, I254C, I254E, I254F, I254G, I254L, I254M, I254N, I254R, I254S, 12454V, I254W, I254Y, I255A, I255L, A256D, L258A, L258D; L258E, L258G, L258H, L258N, L258Q, L258R, L258S, L258T, L258V, D263G, D263K, D263P, D263R, D263S; T264A, T264D, T264G, T2641, T264L, T264N, T264S, D265A, D265G, D265K, D265L, D265N, D265S, D265T, T268A, T268G, T268K, T268L, T268N, T268S, D308A, and Y311E. In one embodiment, the lipase comprises the following amino acid substitutions in comparison to the amino acid sequence of SEQ ID No. 1: S83H, I85L and T268G. In one embodiment, the lipase comprises the following amino acid substitutions in comparison to the amino acid sequence of SEQ ID No. 1: I255A and D265S. In one embodiment, the lipase comprises the following amino acid substitutions in comparison to the amino acid sequence of SEQ ID No. 1: S83H, I254L and D265T. In one embodiment, the lipase comprises the following amino acid substitutions in comparison to the amino acid sequence of SEQ ID No. 1: S83H, I85L and D265T. In one embodiment, the lipase comprises the following amino acid substitutions in comparison to the amino acid sequence of SEQ ID No. 1: S83H, I85L and D265A. In one embodiment, the lipase comprises the following amino acid substitution in comparison to the amino acid sequence of SEQ ID No. 1: I255A. In one embodiment, the lipase comprises the following amino acid substitutions in comparison to the amino acid sequence of SEQ ID No. 1: S83H, I85T and D265S and T268G. In one embodiment, the lipase comprises the following amino acid substitutions in comparison to the amino acid sequence of SEQ ID No. 1: S83H, I85L and T268G. In one embodiment, the lipase has an amino acid sequence according to SEQ ID No. 3. In one embodiment, the lipase is encoded by the nucleic acid sequence according to SEQ ID No. 4.

Further suitable lipases having one or more amino acid substitutions or insertions compared to the sequence according to SEQ ID No. 1 are shown in the following Table 1 wherein LIP062 refers to the lipase according to SEQ ID No. 1.

TABLE 1

| | Amino Acid Residue Position Numbers | | | | | | | | | | | | | | | | | | |
|---|---|---|---|---|---|---|---|---|---|---|---|---|---|---|---|---|---|---|---|
| Lipase | 23 | 33 | 82 | 83 | 84 | 84′ | 85 | 160 | 199 | 254 | 255 | 256 | 258 | 263 | 264 | 265 | 268 | 308 | 311 |
| LIP062 | Y | K | S | S | N | — | I | K | P | I | I | A | L | D | T | D | T | D | Y |
| LIP182 | — | — | — | H | — | — | S | — | — | — | A | — | — | — | A | T | — | — | — |
| LIP181 | — | — | — | H | — | — | V | — | — | — | A | — | — | — | S | T | — | — | — |
| LIP180 | — | — | — | T | — | — | H | — | — | — | A | — | — | — | — | A | — | — | — |
| LIP179 | — | — | — | — | — | — | V | — | — | — | A | — | — | — | S | T | — | — | — |
| LIP178 | — | — | — | H | — | — | L | — | — | — | A | — | — | — | S | A | — | — | — |
| LIP177 | — | — | — | H | — | — | T | — | — | — | A | — | — | — | — | T | — | — | — |
| LIP176 | — | — | — | Y | — | — | A | — | — | — | A | — | — | — | — | T | — | — | — |
| LIP175 | — | — | — | T | — | — | V | — | — | — | A | — | — | — | — | S | — | — | — |
| LIP174 | — | — | — | — | — | — | — | — | — | — | A | — | — | — | — | A | — | — | — |
| LIP173 | — | — | — | — | — | — | — | — | — | — | A | — | — | — | — | S | — | — | — |
| LIP172 | — | — | — | N | — | — | L | — | — | — | A | — | — | — | N | T | — | — | — |
| LIP171 | — | — | — | — | — | — | — | — | — | — | A | — | — | — | D | T | — | — | — |
| LIP170 | — | — | — | N | — | — | L | — | — | — | A | — | — | — | — | T | — | — | — |
| LIP169 | — | — | — | N | — | — | V | — | — | L | — | — | — | — | S | T | — | — | — |
| LIP168 | — | — | — | H | — | — | — | — | — | L | — | — | — | — | A | A | — | — | — |
| LIP167 | — | — | — | H | — | — | — | — | — | L | — | — | — | — | — | T | — | — | — |
| LIP166 | — | — | — | — | — | — | V | — | — | L | — | — | — | — | D | T | — | — | — |
| LIP165 | — | — | — | Y | — | — | — | — | — | — | — | — | — | — | A | T | — | — | — |
| LIP164 | — | — | — | — | — | — | V | — | — | — | — | — | — | — | D | T | — | — | — |
| LIP163 | — | — | — | Y | — | — | A | — | — | — | — | — | — | — | A | T | — | — | — |
| LIP162 | — | — | — | N | — | — | V | — | — | — | — | — | — | — | N | T | — | — | — |
| LIP161 | — | — | — | N | — | — | — | — | — | — | — | — | — | D | T | — | — | — | — |
| LIP160 | — | — | — | H | — | — | L | — | — | — | — | — | — | — | T | — | — | — | — |
| LIP159 | — | — | — | H | — | — | A | — | — | — | — | — | — | A | T | — | — | — | — |
| LIP158 | — | — | — | T | — | — | V | — | — | — | — | — | — | — | T | — | — | — | — |
| LIP157 | — | — | — | H | — | — | L | — | — | — | — | — | — | — | A | — | — | — | — |
| LIP156 | — | — | — | H | — | — | V | — | — | — | — | — | — | — | A | — | — | — | — |
| LIP155 | — | — | — | T | — | — | A | — | — | — | — | — | — | — | T | — | — | — | — |
| LIP154 | — | — | — | H | — | — | V | — | — | — | — | — | — | N | T | — | — | — | — |
| LIP153 | — | — | — | V | — | — | — | — | — | — | — | — | — | — | G | — | — | — | — |
| LIP152 | — | — | — | H | — | — | — | — | — | — | — | — | — | — | A | — | — | — | — |
| LIP151 | — | — | — | Y | — | — | V | — | — | — | — | — | — | S | S | — | — | — | — |
| LIP150 | — | — | — | N | — | — | V | — | — | — | — | — | — | — | G | — | — | — | — |
| LIP149 | — | — | — | H | — | — | — | — | — | — | — | — | — | — | S | — | — | — | — |
| LIP148 | — | — | — | H | — | — | — | — | — | — | — | — | — | — | G | — | — | — | — |
| LIP147 | — | — | — | H | — | — | — | — | — | — | — | — | — | — | S | — | G | — | — |
| LIP146 | — | — | — | H | — | — | — | — | — | — | — | — | — | — | G | — | G | — | — |
| LIP145 | — | — | — | — | — | — | — | — | — | — | — | A | — | — | S | — | G | — | — |
| LIP144 | — | — | — | H | — | — | — | — | — | — | — | A | — | — | G | — | — | — | — |
| LIP143 | — | — | — | H | — | — | — | — | — | — | — | A | — | — | S | — | G | — | — |
| LIP142 | — | — | — | H | — | — | — | — | — | — | — | A | — | — | G | — | G | — | — |
| LIP135 | — | — | — | — | — | — | — | — | — | — | — | L | — | — | — | — | — | — | — |
| LIP134 | — | — | — | — | — | — | — | — | — | — | — | A | — | — | — | — | — | — | — |
| LIP131 | — | — | — | I | — | — | L | — | — | — | — | — | — | — | S | — | — | — | — |
| LIP130 | — | — | — | I | — | — | L | — | — | — | — | — | — | — | G | — | — | — | — |
| LIP126 | — | — | — | — | — | — | — | — | — | — | — | R | — | — | — | — | — | — | — |
| LIP124 | — | — | — | — | — | — | T | — | — | — | — | — | — | — | G | — | G | — | — |
| LIP123 | — | — | — | — | — | — | L | — | — | — | — | — | — | — | G | — | G | — | — |
| LIP120 | — | — | — | H | — | — | L | — | — | — | — | — | — | — | — | — | G | — | — |
| LIP119 | — | — | — | — | — | — | T | — | — | — | — | — | — | — | S | — | G | — | — |
| LIP118 | — | — | — | — | — | — | L | — | — | — | — | — | — | — | G | — | — | — | — |
| LIP117 | — | — | — | — | — | — | T | — | — | — | — | — | — | — | S | — | G | — | — |
| LIP116 | — | — | — | H | — | — | L | — | — | — | — | — | — | — | G | — | G | — | — |
| LIP115 | — | — | — | H | — | — | L | — | — | — | — | — | — | — | S | — | G | — | — |
| LIP114 | — | — | — | H | — | — | L | — | — | — | — | — | — | — | S | — | — | — | — |
| LIP113 | — | — | — | — | — | — | L | — | — | — | — | — | — | — | S | — | G | — | — |
| LIP111 | — | — | — | — | — | — | — | — | — | — | — | — | — | — | A | — | — | — | — |
| LIP110 | — | — | — | — | — | — | — | — | — | — | — | — | — | — | S | — | — | — | — |
| LIP109 | — | — | — | — | — | — | — | — | — | — | — | — | — | — | G | — | — | — | — |
| LIP108 | — | — | — | H | — | — | — | — | — | — | — | — | — | — | — | — | — | — | — |
| LIP102 | — | — | — | — | — | — | T | — | — | — | — | — | — | — | — | — | — | — | — |
| LIP101 | — | — | — | — | — | — | P | — | — | — | — | — | — | — | — | — | — | — | — |
| LIP100 | — | — | — | — | — | — | L | — | — | — | — | — | — | — | — | — | — | — | — |
| LIP099 | — | — | — | — | — | — | A | — | — | — | — | — | — | — | — | — | — | — | — |
| LIP096 | — | — | — | — | — | — | — | — | — | — | — | D | — | — | — | — | — | — | — |
| LIP095 | — | — | — | — | — | — | — | N | — | — | — | — | — | — | — | — | — | — | — |
| LIP094 | — | N | — | — | — | — | — | — | — | — | — | — | — | — | — | — | — | — | — |
| LIP090 | — | — | — | — | — | — | — | — | V | — | — | — | — | — | — | — | — | — | — |
| LIP089 | — | — | T | — | — | — | — | — | — | — | — | — | — | — | — | — | — | — | — |
| LIP062_1909 | — | — | — | — | — | — | T | — | — | — | A | — | — | — | — | — | — | — | — |
| LIP062_1908 | — | — | — | H | — | — | T | — | — | — | A | — | — | — | — | — | — | — | — |
| LIP062_1907 | — | — | — | — | — | — | P | — | — | — | A | — | — | — | — | S | — | — | — |
| LIP062_1906 | — | — | — | H | — | — | P | — | — | — | A | — | — | — | — | — | — | — | — |
| LIP062_1905 | — | — | — | I | — | — | — | — | — | — | A | — | — | — | — | G | G | — | — |
| LIP062_1904 | — | — | — | — | — | — | — | — | — | — | A | — | — | — | — | G | G | — | — |
| LIP062_1903 | — | — | — | H | — | — | P | — | — | — | — | — | — | — | — | — | G | — | — |
| LIP062_1902 | — | — | — | — | — | — | P | — | — | — | — | — | — | — | — | S | G | — | — |
| LIP062_1901 | — | — | — | — | — | — | T | — | — | — | — | — | — | — | — | S | — | — | — |
| LIP062_1900 | — | — | — | H | — | — | T | — | — | — | — | — | — | — | — | — | — | — | — |
| LIP062_1899 | — | — | — | — | — | — | P | — | — | — | — | — | — | — | — | S | — | — | — |
| LIP062_1898 | — | — | — | H | — | — | P | — | — | — | — | — | — | — | — | — | — | — | — |
| LIP062_1897 | — | — | — | I | — | — | — | — | — | — | — | — | — | — | — | G | — | — | — |
| LIP062_1896 | — | — | — | — | — | — | — | — | — | — | — | — | — | — | — | S | G | — | — |
| LIP062_1895 | — | — | — | I | — | — | — | — | — | — | — | — | — | — | — | G | G | — | — |
| LIP062_1894 | — | — | — | — | — | — | — | — | — | — | — | — | — | — | — | G | G | — | — |
| LIP062_1893 | — | — | — | I | — | — | T | — | — | — | — | — | — | — | — | — | G | — | — |
| LIP062_1892 | — | — | — | H | — | — | T | — | — | — | — | — | — | — | — | — | G | — | — |
| LIP062_1891 | — | — | — | I | — | — | T | — | — | — | — | — | — | — | — | S | — | — | — |
| LIP062_1890 | — | — | — | I | — | — | T | — | — | — | — | — | — | — | — | G | — | — | — |
| LIP062_1889 | — | — | — | H | — | — | T | — | — | — | — | — | — | — | — | S | — | — | — |
| LIP062_1888 | — | — | — | H | — | — | T | — | — | — | — | — | — | — | — | G | — | — | — |
| LIP062_1887 | — | — | — | I | — | — | L | — | — | — | — | — | — | — | — | — | G | — | — |
| LIP062_1886 | — | — | — | I | — | — | T | — | — | — | — | — | — | — | — | S | G | — | — |
| LIP062_1885 | — | — | — | I | — | — | L | — | — | — | — | — | — | — | — | S | G | — | — |
| LIP062_1884 | — | — | — | I | — | — | T | — | — | — | — | — | — | — | — | G | G | — | — |
| LIP062_1883 | — | — | — | I | — | — | L | — | — | — | — | — | — | — | — | G | G | — | — |
| LIP062_1882 | — | — | — | H | — | — | T | — | — | — | — | — | — | — | — | G | G | — | — |
| LIP062_1881 | — | — | — | — | — | — | — | — | — | — | — | — | — | — | I | — | — | — | — |
| LIP062_1880 | — | — | — | — | — | — | — | — | — | — | — | — | — | — | L | — | — | — | — |
| LIP062_1879 | — | — | — | — | — | — | — | — | — | — | — | — | — | P | — | — | — | — | — |
| LIP062_1878 | — | — | — | — | — | — | — | — | — | — | — | — | — | G | — | — | — | — | — |
| LIP062_1877 | — | — | — | — | — | — | — | — | — | — | — | — | — | S | — | — | — | — | — |
| LIP062_1876 | — | — | — | — | — | — | — | — | — | — | — | — | — | K | — | — | — | — | — |
| LIP062_1875 | — | — | — | I | N | — | V | — | — | — | — | — | — | — | — | — | — | — | — |
| LIP062_1874 | — | — | — | R | S | — | V | — | — | — | — | — | — | — | — | — | — | — | — |
| LIP062_1873 | — | — | — | — | — | — | — | — | — | — | — | — | — | — | — | — | L | — | — |
| LIP062_1872 | — | — | — | — | — | — | — | — | — | — | — | — | — | — | — | — | A | — | — |
| LIP062_1871 | — | — | — | — | — | — | — | — | — | — | — | — | — | — | — | — | N | — | — |
| LIP062_1870 | — | — | — | — | — | — | — | — | — | — | — | — | — | — | — | — | K | — | — |
| LIP062_1869 | — | — | — | — | — | — | — | — | — | — | — | — | — | — | — | — | S | — | — |
| LIP062_1868 | — | — | — | — | — | — | — | — | — | — | — | — | — | — | — | — | G | — | — |
| LIP062_1867 | — | — | — | — | — | — | — | — | — | — | — | — | — | — | — | L | — | — | — |
| LIP062_1866 | — | — | — | — | — | — | — | — | — | — | — | — | — | — | — | N | — | — | — |
| LIP062_1865 | — | — | — | — | — | — | — | — | — | — | — | — | — | — | — | K | — | — | — |
| LIP062_1864 | — | — | — | N | — | — | — | — | — | — | — | — | — | — | — | — | — | — | — |
| LIP062_1863 | — | — | — | D | — | — | — | — | — | — | — | — | — | — | — | — | — | — | — |
| LIP062_1862 | — | — | — | I | — | — | — | — | — | — | — | — | — | — | — | — | — | — | — |
| LIP062_1861 | A | — | — | — | — | — | — | — | — | — | — | — | — | — | — | — | — | — | — |
| LIP062_1860 | — | — | — | — | — | — | — | — | — | — | — | — | — | — | — | — | — | A | E |
| LIP062_1859 | — | — | — | — | — | — | — | — | — | — | — | — | — | — | — | — | — | A | — |
| LIP062_1858 | — | — | — | — | — | — | — | — | — | — | — | — | — | — | — | — | — | — | E |
| LIP062_1857 | — | — | — | — | — | S | — | — | — | — | — | — | — | — | — | — | — | — | — |
| LIP062_1856 | — | — | — | — | — | L | — | — | — | — | — | — | — | — | — | — | — | — | — |
| LIP062_1855 | — | — | — | — | — | Y | — | — | — | — | — | — | — | — | — | — | — | — | — |
| LIP062_1854 | — | — | — | — | — | — | — | — | — | — | — | — | E | — | — | — | — | — | — |
| LIP062_1853 | — | — | — | — | — | — | — | — | — | — | — | — | Q | — | — | — | — | — | — |
| LIP062_1852 | — | — | — | — | — | — | — | — | — | — | — | — | T | — | — | — | — | — | — |
| LIP062_1851 | — | — | — | — | — | — | — | — | — | — | — | — | H | — | — | — | — | — | — |
| LIP062_1850 | — | — | — | — | — | — | — | — | — | — | — | — | D | — | — | — | — | — | — |
| LIP062_1849 | — | — | — | — | — | — | — | — | — | — | — | — | V | — | — | — | — | — | — |
| LIP062_1848 | — | — | — | — | — | — | — | — | — | — | — | — | R | — | — | — | — | — | — |
| LIP062_1847 | — | — | — | — | — | — | — | — | — | — | — | — | N | — | — | — | — | — | — |
| LIP062_1846 | — | — | — | — | — | — | — | — | — | — | — | — | G | — | — | — | — | — | — |
| LIP062_1845 | — | — | — | — | — | — | — | — | — | — | — | — | A | — | — | — | — | — | — |
| LIP062_1844 | — | — | — | — | — | — | — | — | — | — | — | — | S | — | — | — | — | — | — |
| LIP062_1843 | — | — | — | — | — | — | — | — | — | M | — | — | — | — | — | — | — | — | — |
| LIP062_1842 | — | — | — | — | — | — | — | — | — | G | — | — | — | — | — | — | — | — | — |
| LIP062_1841 | — | — | — | — | — | — | — | — | — | R | — | — | — | — | — | — | — | — | — |
| LIP062_1840 | — | — | — | — | — | — | — | — | — | F | — | — | — | — | — | — | — | — | — |
| LIP062_1839 | — | — | — | — | — | — | — | — | — | E | — | — | — | — | — | — | — | — | — |
| LIP062_1838 | — | — | — | — | — | — | — | — | — | W | — | — | — | — | — | — | — | — | — |
| LIP062_1837 | — | — | — | — | — | — | — | — | — | L | — | — | — | — | — | — | — | — | — |
| LIP062_1836 | — | — | — | — | — | — | — | — | — | Y | — | — | — | — | — | — | — | — | — |
| LIP062_1835 | — | — | — | — | — | — | — | — | — | S | — | — | — | — | — | — | — | — | — |
| LIP062_1834 | — | — | — | — | — | — | — | — | — | C | — | — | — | — | — | — | — | — | — |
| LIP062_1833 | — | — | — | — | — | — | — | — | — | A | — | — | — | — | — | — | — | — | — |
| LIP062_1832 | — | — | — | — | — | — | — | — | — | V | — | — | — | — | — | — | — | — | — |
| LIP062_1831 | — | — | — | — | — | — | — | — | — | N | — | — | — | — | — | — | — | — | — |
| LIP062_1830 | — | — | — | — | — | — | M | — | — | — | — | — | — | — | — | — | — | — | — |
| LIP062_1829 | — | — | — | — | — | — | S | — | — | — | — | — | — | — | — | — | — | — | — |
| LIP062_1828 | — | — | — | — | — | — | C | — | — | — | — | — | — | — | — | — | — | — | — |
| LIP062_1827 | — | N | — | — | — | — | — | N | — | — | — | — | — | — | — | — | — | — | — |
| LIP062_1826 | — | — | — | — | — | — | — | — | I | — | — | — | — | — | — | — | — | — | — |
| LIP062_1825 | — | — | — | N | — | — | V | — | — | — | A | — | — | — | A | G | — | — | — |
| LIP062_1824 | — | — | — | T | — | — | V | — | — | — | A | — | — | — | — | G | — | — | — |
| LIP062_1823 | — | — | — | N | — | — | V | — | — | — | A | — | — | — | S | S | — | — | — |
| LIP062_1822 | — | — | — | H | — | — | T | — | — | — | A | — | — | — | S | S | — | — | — |
| LIP062_1820 | — | — | — | — | — | — | — | — | — | — | A | — | — | — | A | T | — | — | — |
| LIP062_1818 | — | — | — | Y | — | — | — | — | — | — | A | — | — | — | | T | — | — | — |
| LIP062_1817 | — | — | — | — | — | — | — | — | — | — | A | — | — | — | G | T | — | — | — |
| LIP062_1816 | — | — | — | — | — | — | — | — | — | — | A | — | — | — | N | A | — | — | — |
| LIP062_1814 | — | — | — | T | — | — | A | — | — | — | A | — | — | — | — | T | — | — | — |
| LIP062_1812 | — | — | — | N | — | — | — | — | — | — | A | — | — | — | — | A | — | — | — |
| LIP062_1810 | — | — | — | T | — | — | — | — | — | — | A | — | — | — | N | T | — | — | — |
| LIP062_1807 | — | — | — | — | — | — | — | — | — | — | A | — | — | — | D | A | — | — | — |
| LIP062_1805 | — | — | — | H | — | — | V | — | — | — | A | — | — | — | — | A | — | — | — |
| LIP062_1804 | — | — | — | H | — | — | — | — | — | — | A | — | — | — | A | T | — | — | — |
| LIP062_1803 | — | — | — | N | — | — | V | — | — | — | A | — | — | — | S | A | — | — | — |
| LIP062_1801 | — | — | — | — | — | — | — | — | — | — | A | — | — | — | — | G | — | — | — |
| LIP062_1799 | — | — | — | — | — | — | — | — | — | — | A | — | — | — | N | T | — | — | — |
| LIP062_1798 | — | — | — | Y | — | — | V | — | — | — | A | — | — | — | N | T | — | — | — |
| LIP062_1797 | — | — | — | H | — | — | T | — | — | — | A | — | — | — | — | A | — | — | — |
| LIP062_1796 | — | — | — | H | — | — | — | — | — | — | A | — | — | — | A | S | — | — | — |
| LIP062_1795 | — | — | — | N | — | — | V | — | — | — | A | — | — | — | N | T | — | — | — |
| LIP062_1793 | — | — | — | — | — | — | — | — | — | — | A | — | — | — | — | T | — | — | — |
| LIP062_1792 | — | — | — | Y | — | — | V | — | — | — | A | — | — | — | S | T | — | — | — |
| LIP062_1790 | — | — | — | — | — | — | — | — | — | — | A | — | — | — | S | S | — | — | — |
| LIP062_1788 | — | — | — | N | — | — | L | — | — | — | A | — | — | — | S | G | — | — | — |
| LIP062_1782 | — | — | — | N | — | — | — | — | — | L | — | — | — | — | N | T | — | — | — |
| LIP062_1781 | — | — | — | H | — | — | A | — | — | L | — | — | — | — | A | T | — | — | — |
| LIP062_1780 | — | — | — | H | — | — | — | — | — | L | — | — | — | — | — | G | — | — | — |
| LIP062_1779 | — | — | — | N | — | — | V | — | — | L | — | — | — | — | D | T | — | — | — |
| LIP062_1778 | — | — | — | — | — | — | — | — | — | L | — | — | — | — | A | T | — | — | — |
| LIP062_1776 | — | — | — | H | — | — | V | — | — | L | — | — | — | — | — | A | — | — | — |
| LIP062_1775 | — | — | — | T | — | — | V | — | — | L | — | — | — | — | S | A | — | — | — |
| LIP062_1774 | — | — | — | — | — | — | — | — | — | L | — | — | — | — | D | A | — | — | — |
| LIP062_1773 | — | — | — | N | — | — | V | — | — | L | — | — | — | — | A | A | — | — | — |
| LIP062_1770 | — | — | — | — | — | — | — | — | — | L | — | — | — | — | N | T | — | — | — |
| LIP062_1768 | — | — | — | — | — | — | — | — | — | L | — | — | — | — | D | T | — | — | — |
| LIP062_1767 | — | — | — | — | — | — | — | — | — | L | — | — | — | — | S | T | — | — | — |
| LIP062_1766 | — | — | — | — | — | — | — | — | — | L | — | — | — | — | N | A | — | — | — |
| LIP062_1704 | — | — | — | H | — | — | — | — | — | — | — | — | — | — | A | A | — | — | — |
| LIP062_1703 | — | — | — | H | — | — | T | — | — | — | — | — | — | — | A | A | — | — | — |
| LIP062_1701 | — | — | — | T | — | — | V | — | — | — | — | — | — | — | G | T | — | — | — |
| LIP062_1700 | — | — | — | — | — | — | — | — | — | — | — | — | — | — | S | T | — | — | — |
| LIP062_1696 | — | — | — | — | — | — | — | — | — | — | — | — | — | — | A | T | — | — | — |
| LIP062_1695 | — | — | — | N | — | — | V | — | — | — | — | — | — | — | A | T | — | — | — |
| LIP062_1694 | — | — | — | — | — | — | — | — | — | — | — | — | — | — | — | G | — | — | — |
| LIP062_1692 | — | — | — | — | — | — | — | — | — | — | — | — | — | — | A | T | — | — | — |
| LIP062_1691 | — | — | — | N | — | — | V | — | — | — | — | — | — | — | S | S | — | — | — |
| LIP062_1686 | — | — | — | H | — | — | V | — | — | — | — | — | — | — | A | S | — | — | — |
| LIP062_1685 | — | — | — | N | — | — | V | — | — | — | — | — | — | — | N | A | — | — | — |
| LIP062_1684 | — | — | — | — | — | — | — | — | — | — | — | — | — | — | N | T | — | — | — |
| LIP062_1683 | — | — | — | — | — | — | — | — | — | — | — | — | — | — | D | A | — | — | — |
| LIP062_1681 | — | — | — | T | — | — | — | — | — | — | — | — | — | — | N | T | — | — | — |
| LIP062_1680 | — | — | — | N | — | — | A | — | — | — | — | — | — | — | — | T | — | — | — |
| LIP062_1678 | — | — | — | N | — | — | — | — | — | — | — | — | — | — | — | A | — | — | — |
| LIP062_1677 | — | — | — | — | — | — | — | — | — | — | — | — | — | — | G | T | — | — | — |
| LIP062_1676 | — | — | — | Y | — | — | — | — | — | — | — | — | — | — | G | T | — | — | — |
| LIP062_1674 | — | — | — | — | — | — | — | — | — | — | — | — | — | — | G | T | — | — | — |
| LIP062_1670 | — | — | — | N | — | — | — | — | — | — | — | — | — | — | — | T | — | — | — |
| LIP062_1669 | — | — | — | — | — | — | — | — | — | — | — | — | — | — | S | G | — | — | — |
| LIP062_1668 | — | — | — | N | — | — | — | — | — | — | — | — | — | — | — | G | — | — | — |
| LIP062_1667 | — | — | — | — | — | — | A | — | — | — | — | — | — | — | — | G | — | — | — |
| LIP062_1665 | — | — | — | — | — | — | — | — | — | — | — | — | — | — | D | T | — | — | — |
| LIP062_1664 | — | — | — | N | — | — | — | — | — | L | — | — | — | — | A | T | — | — | — |
| LIP062_0450 | — | — | — | — | — | — | F | — | — | — | — | — | — | — | — | — | — | — | — |
| LIP062_0449 | — | — | — | — | — | — | Y | — | — | — | — | — | — | — | — | — | — | — | — |
| LIP062_0391 | — | — | — | — | — | — | — | — | — | — | — | — | — | — | — | — | — | — | — |

The term “cell culture medium” refers to a water-based solution containing one or more chemical compounds that can support the growth of cells.

Preferably, a defined medium is used, i.e. a medium consisting known amounts of chemically defined components as the source of nutrients for the microorganisms. The defined medium may contain various chemically defined nutrient sources selected from the group consisting of a chemically defined hydrogen source, a chemically defined oxygen source, a chemically defined carbon source, a chemically defined nitrogen source, a chemically defined sulfur source, a chemically defined phosphorus source, a chemically defined magnesium source, a chemically defined sodium source, a chemically defined potassium source, a chemically defined trace element source, and a chemically defined vitamin source. Within this description, nutrient sources contained in the defined cell culture medium shall be understood as being chemically defined nutrient sources even if not explicitly mentioned, unless denoted otherwise.

Preferably, the chemically defined carbon source is selected from the group consisting of carbohydrates, organic acids, hydrocarbons, alcohols and mixtures thereof. Preferred carbohydrates are selected from the group consisting of glucose, fructose, galactose, xylose, arabinose, sucrose, maltose, maltotriose, lactose, dextrin, maltodextrins, starch and inulin, and mixtures thereof. Preferred alcohols are selected from the group consisting of glycerol, methanol and ethanol, inositol, mannitol and sorbitol and mixtures thereof. Preferred organic acids are selected from the group consisting of acetic acid, propionic acid, lactic acid, formic acid, malic acid, citric acid, fumaric acid and higher alkanoic acids and mixtures thereof. Preferably, the chemically defined carbon source comprises glucose or sucrose. More preferably, the chemically defined carbon source comprises glucose.

A first carbon source is used in the first phase of the culturing process of the present invention. The first carbon source is part of the cell culture medium in which the cells are cultured for a time period. In the fed-batch method the cells are cultured in the cell culture medium comprising the first carbon source until the feeding is initiated. Preferably, the first carbon source is glucose. The concentration of the first carbon source in the cell culture medium is 5 to 30 g/l, preferably 8 to 25 g/l, more preferably 10 to 22 g/l, even more preferably 12 to 20 g/l and most preferably 13 to 18 g/l. The concentration of glucose in the cell culture medium is 5 to 30 g/l, preferably 8 to 25 g/l, more preferably 10 to 22 g/l, even more preferably 12 to 20 g/l and most preferably 13 to 18 g/l.

In the method of the present invention the second carbon source is fed to the first culture when the first carbon source is depleted, i.e. when the first carbon source is not detectable anymore. Preferably, the depletion of the first carbon source is indicated by a rapid decrease in oxygen uptake measured by dissolved oxygen. Methods for measuring the content of a carbon source such as glucose in a cell culture are well-known and include colorimetric or fluorometric methods. Suitable kits for measuring the content of a carbon source such as glucose are commercially available. In one embodiment the content of a carbon source such as glucose in the cell culture is measured online using a suitable apparatus such as an YSI analyzer.

The second carbon source may be fed as a part of a feeding medium or it may be fed as a solution of the second carbon source without further compounds. Preferably, the second carbon source is fed as a solution of the second carbon source which does not contain further compounds. The second and the first carbon source may be the same or different. Preferably, the first and second carbon source are the same. Also, preferably, the second carbon source is glucose. More preferably, the glucose is fed as a glucose solution. Even more preferably, the glucose solution has a glucose concentration of 500 to 900 g/l such as 700 g/l.

The chemically defined nitrogen source is preferably selected from the group consisting of urea, ammonia, nitrate, nitrate salts, nitrit, ammonium salts such as ammonium chloride, ammonium sulfate, ammonium acetate, ammonium phosphate and ammonium nitrate, and amino acids such as glutamate or lysine and combinations thereof. More preferably, a chemically defined nitrogen source is selected from the group consisting of ammonia, ammonium sulfate and ammonium phosphate. Even more preferably, the cell culture medium contains ammonium sulfate.

The ammonium concentration in the cell culture medium is 5 mM to 80 mM, preferably the ammonium concentration in the cell culture medium is 5 mM to 75 mM or 6 mM to 70 mM or 6 to 65 mM, more preferably the ammonium concentration in the cell culture medium is 7 mM to 60 mM or 7 mM to 55 mM or 7 mM to 50 mM, even more preferably the ammonium concentration in the cell culture medium is 8 mM to 45 mM or 8 mM to 40 mM or 9 mM to 35 mM or 9 mM to 30 mM and most preferably the ammonium concentration in the cell culture medium is 10 mM to 25 mM or 10 mM to 20 mM.

Oxygen is usually provided during the cultivation of the cells by aeration of the fermentation media by stirring or gassing. Hydrogen is usually provided due to the presence of water in the aqueous cell culture medium. However, hydrogen and oxygen are also contained within the chemically defined carbon and/or chemically defined nitrogen source and can be provided that way.

Magnesium can be provided to the cell culture medium in chemically defined form by one or more magnesium salts, preferably selected from the group consisting of magnesium chloride, magnesium sulfate, magnesium nitrate, magnesium phosphate, and combinations thereof, or by magnesium hydroxide, or by combinations of one or more magnesium salts and magnesium hydroxide. Preferably, the magnesium salt is magnesium sulfate. Preferably, the magnesium concentration in the cell culture medium is 5 mM to 30 mM or 5 mM to 28 mM or 6 mM to 25 mM or 6 mM to 22 mM, more preferably the magnesium concentration in the cell culture medium is 7 mM to 20 mM or 8 mM to 20 mM or 9 mM to 20 mM, even more preferably the magnesium concentration in the cell culture medium is 10 mM to 18 mM or 11 mM to 17 mM and most preferably the magnesium concentration in the cell culture medium is 12 mM to 16 mM or 13 mM to 15 mM.

Calcium can be added to the cell culture medium by one or more calcium salts, preferably selected from the group consisting of calcium sulfate, calcium chloride, calcium nitrate, calcium phosphate, calcium hydroxide, and combination thereof. Preferably, the calcium salt is calcium sulfate. Preferably, the calcium concentration in the cell culture medium is 0.5 mM to 3 mM or 0.6 mM to 2.8 mM or 0.7 mM to 2.7 mM, more preferably the calcium concentration in the cell culture medium is 0.8 mM to 2.6 mM or 0.9 mM to 2.5 mM or 1.0 mM to 2.4 mM, even more preferably the calcium concentration in the cell culture medium is 1.1 mM to 2.3 mM or 1.2 mM to 2.1 mM or 1.3 mM to 2.0 mM and most more preferably the calcium concentration in the cell culture medium is 1.4 mM to 1.9 mM or 1.5 mM to 1.8 mM.

Potassium can be added to the cell culture medium in chemically defined form by one or more potassium salts, preferably selected from the group consisting of potassium chloride, potassium nitrate, potassium sulfate, potassium phosphate, potassium hydroxide, and combination thereof. Preferably, the cell culture medium contains potassium phosphate. The potassium concentration in the cell culture medium is 80 mM to 250 mM or 90 mM to 240 mM, preferably the potassium concentration in the cell culture medium is 100 mM to 230 mM or 110 mM to 220 mM, more preferably the potassium concentration in the cell culture medium is 120 mM to 210 mM or 130 mM to 200 mM or 140 mM to 190 mM and most preferably the potassium concentration in the cell culture medium is 150 mM to 170 mM.

Phosphorus can be added to the cell culture medium in chemically defined form by one or more salts comprising phosphorus, preferably selected from the group consisting of potassium phosphate, sodium phosphate, magnesium phosphate, phosphoric acid, and combinations thereof. Preferably, the cell culture medium contains potassium phosphate. The phosphate concentration in the cell culture medium is 10 mM to 150 mM or 20 mM to 130 mM, preferably the phosphate concentration in the cell culture medium is 30 mM to 120 mM or 40 mM to 100 mM, more preferably the phosphate concentration in the cell culture medium is 50 mM to 90 mM or 55 mM to 85 mM and most preferably the phosphate concentration in the cell culture medium is 60 mM to 80 mM.

Sulfur can be added to the cell culture medium in chemically defined form by one or more salts comprising sulfur, preferably selected from the group consisting of potassium sulfate, sodium sulfate, calcium sulfate, magnesium sulfate, sulfuric acid, and combinations thereof. Preferably, the cell culture medium contains calcium sulfate. The sulfate concentration in the cell culture medium is 5 mM to 100 mM or 10 mM to 90 mM, preferably the sulfate concentration in the cell culture medium is 15 mM to 85 mM or 20 mM to 80 mM, more preferably the sulfate concentration in the cell culture medium is 30 mM to 75 mM or 35 mM to 70 mM and most preferably the sulfate concentration in the cell culture medium is 40 mM to 65 mM or 45 mM to 55 mM.

The present inventors have found that using a cell culture medium which has a higher concentration of potassium ions relative to the concentration of sulfate ions and/or phosphate ions increases product titer.

Hence, in one embodiment the molar ratio of potassium ions to phosphate ions is 1.5:1 to 2.8:1 or 1.6:1 to 2.7:1, preferably the molar ratio of potassium ions to phosphate ions is 1.7:1 to 2.6:1 or 1.8:1 to 2.6:1, more preferably the molar ratio of potassium ions to phosphate ions is 1.9:1 to 2.5:1 or 2.0:1 to 2.5:1 and most preferably the molar ratio of potassium ions to phosphate ions is 2.1:1 to 2.4:1 or 2.3:1.

Hence, in one embodiment the molar ratio of potassium ions to sulfate ions is 2:1 to 4:1 or 2.3:1 to 4:1, preferably the molar ratio of potassium ions to sulfate ions is 2.3:1 to 3.8:1 or 2.5:1 to 3.8:1, more preferably the molar ratio of potassium ions to sulfate ions is 2:7:1 to 3.6:1 or 2.8:1 to 3.5:1 and most preferably the molar ratio of potassium ions to sulfate ions is 3.0:1 to 3.4:1 or 3.2:1.

In one embodiment, the molar ratio of potassium ions to phosphate ions is 1.5:1 to 2.8:1 or 1.6:1 to 2.7:1 and the molar ratio of potassium ions to sulfate ions is 2:1 to 4:1 or 2.3:1 to 4:1, preferably the molar ratio of potassium ions to phosphate ions is 1.7:1 to 2.6:1 or 1.8:1 to 2.6:1 and the molar ratio of potassium ions to sulfate ions is 2.3:1 to 3.8:1 or 2.5:1 to 3.8:1, more preferably the molar ratio of potassium ions to phosphate ions is 1.9:1 to 2.5:1 or 2.0:1 to 2.5:1 and the molar ratio of potassium ions to sulfate ions is 2:7:1 to 3.6:1 or 2.8:1 to 3.5:1, even more preferably the molar ratio of potassium ions to phosphate ions is 2.1:1 to 2.4:1 and the molar ratio of potassium ions to sulfate ions is 3.0:1 to 3.4:1 and most preferably the molar ratio of potassium ions to phosphate ions is 2.3:1 and the molar ratio of potassium ions to sulfate ions is 3.2:1.

In one embodiment, the cell culture medium contains:
80 mM to 250 mM or 90 mM to 240 mM potassium and
10 mM to 150 mM or 20 mM to 130 mM phosphate and
5 mM to 100 mM or 10 mM to 90 mM sulfate.

In one embodiment, the cell culture medium contains:
100 mM to 230 mM or 110 mM to 220 mM potassium and
30 mM to 120 mM or 40 mM to 100 mM phosphate and
15 mM to 85 mM or 20 mM to 80 mM sulfate.

In one embodiment, the cell culture medium contains:
120 mM to 210 mM or 130 mM to 200 mM or 140 mM to 190 mM potassium and
50 mM to 90 mM or 55 mM to 85 mM phosphate and
30 mM to 75 mM or 35 mM to 70 mM sulfate.

In one embodiment, the cell culture medium contains:
150 mM to 170 mM potassium and
60 mM to 80 mM phosphate and
40 mM to 65 mM or 45 mM to 55 mM sulfate.

In one embodiment, the cell culture medium contains:
165 mM to 168 mM potassium and
70 mM to 75 mM phosphate and
50 mM to 53 mM sulfate.

In one embodiment, the cell culture medium contains:
80 mM to 250 mM or 90 mM to 240 mM potassium and
10 mM to 150 mM or 20 mM to 130 mM phosphate and
5 mM to 100 mM or 10 mM to 90 mM sulfate and
5 mM to 80 mM or 5 mM to 75 mM or 6 mM to 70 mM or 6 to 65 mM ammonium.

In one embodiment, the cell culture medium contains:
100 mM to 230 mM or 110 mM to 220 mM potassium and
30 mM to 120 mM or 40 mM to 100 mM phosphate and
15 mM to 85 mM or 20 mM to 80 mM sulfate and
7 mM to 60 mM or 7 mM to 55 mM or 7 mM to 50 mM ammonium.

In one embodiment, the cell culture medium contains:
120 mM to 210 mM or 130 mM to 200 mM or 140 mM to 190 mM potassium and
50 mM to 90 mM or 55 mM to 85 mM phosphate and
30 mM to 75 mM or 35 mM to 70 mM sulfate and
8 mM to 45 mM or 8 mM to 40 mM or 9 mM to 35 mM or 9 mM to 30 mM ammonium.

In one embodiment, the cell culture medium contains:
150 mM to 170 mM potassium and
60 mM to 80 mM phosphate and
40 mM to 65 mM or 45 mM to 55 mM sulfate and
10 mM to 25 mM ammonium.

In one embodiment, the cell culture medium contains:
165 mM to 168 mM potassium and
70 mM to 75 mM phosphate and
50 mM to 53 mM sulfate and
10 mM to 15 mM ammonium.

In one embodiment, the cell culture medium contains:
80 mM to 250 mM or 90 mM to 240 mM potassium and
10 mM to 150 mM or 20 mM to 130 mM phosphate and
5 mM to 100 mM or 10 mM to 90 mM sulfate and
5 mM to 80 mM or 5 mM to 75 mM or 6 mM to 70 mM or 6 to 65 mM ammonium and
5 mM to 30 mM or 5 mM to 28 mM or 6 mM to 25 mM or 6 mM to 22 mM magnesium.

In one embodiment, the cell culture medium contains:
100 mM to 230 mM or 110 mM to 220 mM potassium and
30 mM to 120 mM or 40 mM to 100 mM phosphate and
15 mM to 85 mM or 20 mM to 80 mM sulfate and
7 mM to 60 mM or 7 mM to 55 mM or 7 mM to 50 mM ammonium and
7 mM to 20 mM or 8 mM to 20 mM or 9 mM to 20 mM magnesium.

In one embodiment, the cell culture medium contains:
120 mM to 210 mM or 130 mM to 200 mM or 140 mM to 190 mM potassium and
50 mM to 90 mM or 55 mM to 85 mM phosphate and
30 mM to 75 mM or 35 mM to 70 mM sulfate and
8 mM to 45 mM or 8 mM to 40 mM or 9 mM to 35 mM or 9 mM to 30 mM ammonium and
10 mM to 18 mM or 11 mM to 17 mM magnesium.

In one embodiment, the cell culture medium contains:
150 mM to 170 mM potassium and
60 mM to 80 mM phosphate and
40 mM to 65 mM or 45 mM to 55 mM sulfate and
10 mM to 25 mM or 10 mM to 20 mM ammonium and
12 mM to 16 mM or 13 mM to 15 mM magnesium.

In one embodiment, the cell culture medium contains:
165 mM to 168 mM potassium and
70 mM to 75 mM phosphate and
50 mM to 53 mM sulfate and
10 mM to 15 mM ammonium and
14 to 15 mM magnesium.

In one embodiment, the cell culture medium contains:
80 mM to 250 mM or 90 mM to 240 mM potassium and
10 mM to 150 mM or 20 mM to 130 mM phosphate and
5 mM to 100 mM or 10 mM to 90 mM sulfate and
5 mM to 80 mM or 5 mM to 75 mM or 6 mM to 70 mM or 6 to 65 mM ammonium and
5 mM to 30 mM or 5 mM to 28 mM or 6 mM to 25 mM or 6 mM to 22 mM magnesium and
0.5 mM to 3 mM or 0.6 mM to 2.8 mM or 0.7 mM to 2.7 mM calcium.

In one embodiment, the cell culture medium contains:
100 mM to 230 mM or 110 mM to 220 mM potassium and
30 mM to 120 mM or 40 mM to 100 mM phosphate and
15 mM to 85 mM or 20 mM to 80 mM sulfate and
7 mM to 60 mM or 7 mM to 55 mM or 7 mM to 50 mM ammonium and
7 mM to 20 mM or 8 mM to 20 mM or 9 mM to 20 mM magnesium and
0.8 mM to 2.6 mM or 0.9 mM to 2.5 mM or 1.0 mM to 2.4 mM calcium.

In one embodiment, the cell culture medium contains:
120 mM to 210 mM or 130 mM to 200 mM or 140 mM to 190 mM potassium and
50 mM to 90 mM or 55 mM to 85 mM phosphate and
30 mM to 75 mM or 35 mM to 70 mM sulfate and
8 mM to 45 mM or 8 mM to 40 mM or 9 mM to 35 mM or 9 mM to 30 mM ammonium and
10 mM to 18 mM or 11 mM to 17 mM magnesium and
1.1 mM to 2.3 mM or 1.2 mM to 2.1 mM or 1.3 mM to 2.0 mM calcium.

In one embodiment, the cell culture medium contains:
150 mM to 170 mM potassium and
60 mM to 80 mM phosphate and
40 mM to 65 mM or 45 mM to 55 mM sulfate and
10 mM to 25 mM or 10 mM to 20 mM ammonium and
12 mM to 16 mM or 13 mM to 15 mM magnesium and
1.4 mM to 1.9 mM or 1.5 mM to 1.8 mM calcium.

In one embodiment, the cell culture medium contains:
165 mM to 168 mM potassium and
70 mM to 75 mM phosphate and
50 mM to 53 mM sulfate and
10 mM to 15 mM ammonium and
14 to 15 mM magnesium and
1.7 mM to 1.8 mM calcium.

One or more trace element ions can be added to the cell culture medium in chemically defined form, preferably in amounts of below 10 mmol/L initial cell culture medium each. These trace element ions are selected from the group consisting of iron, copper, manganese, zinc, cobalt, nickel, molybdenum, selenium, and boron and combinations thereof. Preferably, the trace element ions iron, copper, manganese and zinc are added to the cell culture medium. Preferably, the one or more trace element ions are added to the cell culture medium in an amount selected from the group consisting of 50 μmol to 5 mmol per liter of initial medium of iron, 40 μmol to 4 mmol per liter of initial medium copper, 30 μmol to 3 mmol per liter of initial medium manganese and 20 μmol to 2 mmol per liter of initial medium zinc, and combinations thereof. For adding each trace element preferably one or more from the group consisting of chloride, phosphate, sulfate, nitrate, citrate and acetate salts can be used. Preferably, the sulfate salts are used. More preferably, iron sulfate, zinc sulfate, manganse sulfate and copper sulfate are added to the cell culture medium.

Compounds which may optionally be included in a chemically defined medium are chelating agents, such as citric acid, MGDA, NTA, or GLDA, and buffering agents such as mono- and dipotassium phosphate, calcium carbonate, and the like. Buffering agents preferably are added when dealing with processes without an external pH control. In addition, an antifoaming agent may be dosed prior to and/or during the fermentation process.

Vitamins refer to a group of structurally unrelated organic compounds, which are necessary for the normal metabolism of cells. Cells are known to vary widely in their ability to synthesize the vitamins they require. Vitamins can be selected from the group of thiamin, riboflavin, pyridoxal, nicotinic acid or nicotinamide, pantothenic acid, cyanocobalamin, folic acid, biotin, lipoic acid, purines, pyrimidines, inositol, choline and hemins. Preferably, the cell culture medium comprises biotin.

In one embodiment the cell culture medium used in the method of the present invention does not comprise urea or a derivative thereof such as dimethylurea, diethylurea, N-acetylphenyl urea, isopropyliden urea and phenyl urea.

The present inventors have further found that a temperature shift from a first temperature to a second temperature increases the titer of the recombinant protein. Preferably, the second temperature is lower than the first temperature. More preferably, the second temperature is 1 to 8° C. lower than the first temperature. Even more preferably, the second temperature is 2 to 7° C. or 3 to 5° C. lower than the first temperature. Most preferably, the second temperature is 4° C. lower than the first temperature.

The first temperature is between 28° C. and 37° C., preferably between 29° C. and 35° C., more preferably between 29° C. and 33° C. or between 29° C. and 31° C. and most preferably the first temperature is 30° C.

The second temperature is between 23° C. and 30° C., preferably between 24° C. and 29° C., more preferably between 25° C. and 28° C. and most preferably the second temperature is 26° C.

In one embodiment, the first temperature is between 28° C. and 37° C. and the second temperature is between 23° C. and 30° C., wherein the second temperature is lower than the first temperature. In one embodiment, the first temperature is between 29° C. and 35° C. and the second temperature is between 24° C. and 29° C., wherein the second temperature is lower than the first temperature. In one embodiment, the first temperature is between 29° C. and 33° C. and the second temperature is between 25° C. and 28° C. In one embodiment, the first temperature is 30° C. and the second temperature is 26° C.

Preferably, the first temperature is changed to the second temperature linearly over a period of time, i.e. the temperature decreases from the first temperature to the second temperature in a linear manner. A linear temperature shift can be accomplished using an automation control software and automated heating or cooling. In one embodiment the first temperature is changed to the second temperature linearly over a period of 30 minutes to two hours, preferably the first temperature is changed to the second temperature linearly over a period of 40 minutes to 100 minutes, more preferably the first temperature is changed to the second temperature linearly over a period of 50 minutes to 80 minutes and most preferably the first temperature is changed to the second temperature linearly over a period of one hour.

The first temperature is changed to the second temperature at the time the feeding of the second carbon source is initiated, i.e. at the time the second carbon source is added to the first culture for the first time. If the temperature is changed linearly from the first temperature to the second temperature over a period of time, the temperature change is initiated at the time the feeding of the second carbon source is initiated, i.e. at the time the second carbon source is added to the first culture for the first time. The second temperature is maintained until the culturing is stopped and the recombinant protein is obtained.

In one embodiment the method of the present invention further comprises a pH shift from a first culture pH to a second culture pH. Preferably, the second pH is higher than the first pH. More preferably, the second pH is 0.5 to 2 pH units higher than the first pH. Even more preferably, the second pH is 0.7 to 1.5 pH units or 0.8 to 1.3 pH units higher than the first pH. Most preferably, the second pH is 1 pH unit higher than the first pH.

The first pH is between 4.0 and 6.0, preferably between 4.2 and 5.7, more preferably between 4.5 and 5.5 or between 4.8 and 5.2 and most preferably the first pH is 5.0.

The second pH is between 5.0 and 7.0, preferably between 5.2 and 6.7, more preferably between 5.5 and 6.5 or between 5.8 and 6.2 and most preferably the second pH is 6.0.

In one embodiment, the first pH is between 4.0 and 6.0 and the second pH is between 5.0 and 7.0, wherein the second pH is higher than the first pH. In one embodiment, the first pH is between 4.2 and 5.7 and the second pH is between 5.2 and 6.7, wherein the second pH is higher than the first pH. In one embodiment, the first pH is between 4.8 and 5.2 and the second pH is between 5.5 and 6.5 or the first pH is between 4.5 and 5.5 and the second pH is between 5.8 and 6.2. In one embodiment, the first pH is 5.0 and the second pH is 6.0.

In one embodiment, the first temperature is between 28° C. and 37° C. and the second temperature is between 23° C. and 30° C., wherein the second temperature is lower than the first temperature, and the first pH is between 4.0 and 6.0 and the second pH is between 5.0 and 7.0, wherein the second pH is higher than the first pH. In one embodiment, the first temperature is between 29° C. and 35° C. and the second temperature is between 24° C. and 29° C., wherein the second temperature is lower than the first temperature, and the first pH is between 4.2 and 5.7 and the second pH is between 5.2 and 6.7, wherein the second pH is higher than the first pH. In one embodiment, the first temperature is between 29° C. and 33° C. and the second temperature is between 25° C. and 28° C. and the first pH is between 4.8 and 5.2 and the second pH is between 5.5 and 6.5 or the first pH is between 4.5 and 5.5 and the second pH is between 5.8 and 6.2. In one embodiment, the first temperature is 30° C. and the second temperature is 26° C. and the first pH is 5.0 and the second pH is 6.0.

Preferably, the first pH is changed to the second pH linearly over a period of time, i.e. the pH increases from the first pH to the second pH in a linear manner. A linear pH shift can be accomplished using an automation control software which controls the addition of ammonium hydroxide solution. In one embodiment the first pH is changed to the second pH linearly over a period of 30 minutes to two hours, preferably the first pH is changed to the second pH linearly over a period of 40 minutes to 100 minutes, more preferably the first pH is changed to the second pH linearly over a period of 50 minutes to 80 minutes and most preferably the first pH is changed to the second pH linearly over a period of one hour.

The first pH is changed to the second pH at the time the feeding of the second carbon source is initiated, i.e. at the time the second carbon source is added to the first culture for the first time. If the pH is changed linearly from the first pH to the second pH over a period of time, the pH change is initiated at the time the feeding of the second carbon source is initiated, i.e. at the time the second carbon source is added to the first culture for the first time. The second pH is maintained until the culturing is stopped and the recombinant protein is obtained.

The feeding of the second carbon source may be a pulse feeding, i.e. discrete amounts of the second carbon source are added and between these additions no second carbon source is added to the first culture. The pulse feeding may be initiated when the level of dissolved oxygen exceeds a certain threshold. In another embodiment, the feeding of the second carbon source is continuous, i.e. the feeding is not interrupted and the second carbon source is delivered to the first culture continuously at a specific feed rate. In a preferred embodiment, the feeding of the second carbon source is continuous. The present inventors have found that by using continuous feeding the predictability between different production scales, in particular between lab scale and pilot scale, is improved.

In one embodiment, the continuous feeding of the second carbon source comprises initiating feeding at an initial feed rate and increasing the feed rate exponentially to a maximum feed rate. After the maximum feed rate is reached, the feed rate is decreased to an intermediate feed rate. In one embodiment the decrease from the maximum feed rate to the intermediate feed rate is linear. After the intermediate feed rate is reached, the feed rate is further decreased to the final feed rate which is maintained until the culturing is stopped and the recombinant protein is obtained.

In one embodiment, the initial feed rate is 2 to 6 g/l/h of the second carbon source, preferably the initial feed rate is 2.5 to 5 g/l/h of the second carbon source, more preferably the initial feed rate is 3 to 4 g/l/h of the second carbon source and most preferably the initial feed rate is 3.5 g/l/h of the second carbon source.

In one embodiment, the initial feed rate is increased with a rate of 0.05 to 0.3 per hour, preferably with a rate of 0.1 to 0.25 per hour, more preferably with a rate of 0.12 to 0.2 per hour and most preferably with a rate of 0.15 per hour.

In one embodiment, the maximum feed rate is 15 to 22 g/l/h of the second carbon source, preferably the maximum feed rate is 16 to 21 g/l/h of the second carbon source, more preferably the maximum feed rate is 17 to 20 g/l/h of the second carbon source, even more preferably the maximum feed rate is 18 to 19 g/l/h of the second carbon source and most preferably the maximum feed rate is 18.8 g/l/h of the second carbon source.

In one embodiment, the intermediate feed rate is higher than the initial feed rate. In one embodiment, the intermediate feed rate is 5 to 8 g/l/h of the second carbon source, preferably the intermediate feed rate is 5.5 to 7.5 g/l/h of the second carbon source, more preferably the intermediate feed rate is 6 to 7 g/l/h of the second carbon source and most preferably the intermediate feed rate is 6.5 g/l/h of the second carbon source.

In one embodiment, the final feed rate is higher than the initial feed rate. In one embodiment, the final feed rate is 2 to 6 g/l/h of the second carbon source, preferably the final feed rate is 2.5 to 5 g/l/h of the second carbon source, more preferably the final feed rate is 3 to 4 g/l/h of the second carbon source and most preferably the final feed rate is 3.8 g/l/h of the second carbon source.

In one embodiment, the initial feed rate is 2 to 6 g/l/h of the second carbon source, the initial feed rate is increased with a rate of 0.05 to 0.3 per hour, the maximum feed rate is 15 to 22 g/l/h of the second carbon source, the intermediate feed rate is 5 to 8 g/l/h of the second carbon source and the final feed rate is 2 to 6 g/l/h of the second carbon source.

In one embodiment, the initial feed rate is 2.5 to 5 g/l/h of the second carbon source, the initial feed rate is increased with a rate of 0.1 to 0.25 per hour, the maximum feed rate is 16 to 21 g/l/h of the second carbon source, the intermediate feed rate is 5.5 to 7.5 g/l/h of the second carbon source and the final feed rate is 2.5 to 5 g/l/h of the second carbon source.

In one embodiment, the initial feed rate is 3 to 4 g/l/h of the second carbon source, the initial feed rate is increased with a rate of 0.12 to 0.2 per hour, the maximum feed rate is 17 to 20 g/l/h of the second carbon source, the intermediate feed rate is 6 to 7 g/l/h of the second carbon source and the final feed rate is 3 to 4 g/l/h of the second carbon source.

In one embodiment, the initial feed rate is 3.5 g/l/h of the second carbon source, the initial feed rate is increased with a rate of 0.15 per hour, the maximum feed rate is 18.8 g/l/h of the second carbon source, the intermediate feed rate is 6.5 g/l/h of the second carbon source and the final feed rate is 3.8 g/l/h of the second carbon source.

In one embodiment, the dissolved oxygen content during the culturing and feeding steps of the method is maintained at 15% to 25% of saturation, preferably the dissolved oxygen content during the culturing and feeding steps of the method is maintained at 16% to 22% of saturation, more preferably the dissolved oxygen content during the culturing and feeding steps of the method is maintained at 17% to 19% of saturation and most preferably the dissolved oxygen content during the culturing and feeding steps of the method is maintained at 18% of saturation.

The culturing is initiated by inoculating the cell culture medium with a seed culture of yeast cells. In one embodiment, the cell culture medium is inoculated with 5 to 15% v/v of the seed culture, preferably with 6 to 13% v/v of the seed culture, more preferably with 8 to 12% v/v of the seed culture and most preferably with 10% v/v of the seed culture.

The culture period is defined as the period from inoculating the cell culture medium with a seed culture of yeast cells until the recombinant protein is obtained. Accordingly, the culture period is the period in which both step (a) of culturing the yeast cells and step (b) of feeding a second carbon source to said first culture are performed. The culture period is from 90 to 150 hours, preferably from 100 to 140 hours, more preferably from 110 to 130 hours and most preferably the culture period is 120 hours.

The protein produced by the host cell may be obtained by any known process for isolating and purifying proteins. Such processes include, but are not limited to, salting out and solvent precipitation, ultrafiltration, gel electrophoresis, ion-exchange chromatography, affinity chromatography, reverse phase high performance liquid chromatography, hydrophobic interaction chromatography, mixed mode chromatography, hydroxyapatite chromatography and isoelectric focusing.

The titer of the recombinant protein in the supernatant of the recombinant yeast cells is from 1 to 20 g/l or from 1.5 to 20 g/l or from 2.0 to 20 g/l or from 2.5 to 20 g/l or from 3.0 to 20 g/l or from 3.5 to 20 g/l or from 4.0 to 20 g/l or from 4.5 to 20 g/l or from 5.0 to 20 g/l or from 5.5 to 20 g/I or from 6.0 to 20 g/l or from 6.5 to 20 g/l or from 7.0 to 20 g/l or from 7.5 to 20 g/l or from 8.0 to 20 g/l or from 8.5 to 20 g/l. The titer of the recombinant protein in the supernatant of the recombinant yeast cells is from 6 g/l to 20 g/I or from 6 g/l to 19.0 g/l or from 6 g/l to 18.5 g/l or from 6 g/l to 18.0 g/l or from 6 g/l to 17.5 g/l or from 6 g/l to 17.0 g/l or from 6 g/l to 16.5 g/l or from 6 g/l to 16.0 g/l or from 6 g/l to 15.5 g/l or from 6 g/l to 15.0 g/l or from 6 g/l to 14.5 g/l or from 6 g/l to 14.0 g/l. The titer of the recombinant protein in the supernatant of the recombinant yeast cells is from 6.5 to 13.5 g/l or from 7.0 to 13.0 g/l or from 7.5 to 12.5 g/l or from 8 to 12 g/l.

Some specific embodiments of the present invention relate to:
1. A method for producing a recombinant protein in Pichia pastoris cells, comprising the steps of:
   (a) culturing yeast cells genetically modified to express the recombinant protein in a suitable cell culture medium comprising a first carbon source at a first temperature of 28° C. and 37° C., thereby obtaining a first culture;
   (b) feeding a second carbon source to said first culture when the first carbon source is depleted,
   wherein the first temperature is changed to a second temperature of 23° C. and 30° C. at the time the feeding of step (b) is initiated, wherein the second temperature is lower than the first temperature; and
   (c) obtaining the recombinant protein.
2. A method for producing a recombinant protein in Pichia pastoris cells, comprising the steps of:
   (a) culturing yeast cells genetically modified to express the recombinant protein in a suitable cell culture medium comprising a first carbon source at a first temperature of 30° C., thereby obtaining a first culture;
   (b) feeding a second carbon source to said first culture when the first carbon source is depleted,
   wherein the first temperature is changed to a second temperature of 26° C. at the time the feeding of step (b) is initiated; and
   (c) obtaining the recombinant protein.
3. A method for producing a recombinant protein in Pichia pastoris cells, comprising the steps of:
   (a) culturing yeast cells genetically modified to express the recombinant protein in a suitable cell culture medium comprising a first carbon source at a first temperature of 28° C. and 37° C. and a first pH between 4.0 and 6.0, thereby obtaining a first culture;
   (b) feeding a second carbon source to said first culture when the first carbon source is depleted,
   wherein the first temperature is changed to a second temperature of 23° C. and 30° C. at the time the feeding of step (b) is initiated, wherein the second temperature is lower than the first temperature and wherein the first pH is changed to a second pH of 5.0 to 7.0 at the time the feeding of step (b) is initiated, wherein the second pH is higher than the first pH; and
   (c) obtaining the recombinant protein.
4. A method for producing a recombinant protein in Pichia pastoris cells, comprising the steps of:
   (a) culturing yeast cells genetically modified to express the recombinant protein in a suitable cell culture medium comprising a first carbon source at a first temperature of 30° C. and a first pH of 5.0, thereby obtaining a first culture;
   (b) feeding a second carbon source to said first culture when the first carbon source is depleted,
   wherein the first temperature is changed to a second temperature of 26° C. at the time the feeding of step (b) is initiated and the first pH is changed to a second pH of 6.0 at the time the feeding of step (b) is initiated; and
   (c) obtaining the recombinant protein.
5. A method for producing a recombinant protein in Pichia pastoris cells, comprising the steps of:
   (a) culturing yeast cells genetically modified to express the recombinant protein in a suitable cell culture medium with a molar ratio of potassium ions to phosphate ions is 1.5:1 to 2.8:1 or 1.6:1 to 2.7:1 and a molar ratio of potassium ions to sulfate ions is 2:1 to 4:1 or 2.3:1 to 4:1 and comprising a first carbon source at a first temperature of 28° C. and 37° C., thereby obtaining a first culture;
   (b) feeding a second carbon source to said first culture when the first carbon source is depleted,
   wherein the first temperature is changed to a second temperature of 23° C. and 30° C. at the time the feeding of step (b) is initiated, wherein the second temperature is lower than the first temperature; and
   (c) obtaining the recombinant protein.
6. A method for producing a recombinant protein in Pichia pastoris cells, comprising the steps of:
   (a) culturing yeast cells genetically modified to express the recombinant protein in a suitable cell culture medium with a molar ratio of potassium ions to phosphate ions is 2.3:1 and a molar ratio of potassium ions to sulfate ions is 3.2:1 and comprising a first carbon source at a first temperature of 30° C., thereby obtaining a first culture;
   (b) feeding a second carbon source to said first culture when the first carbon source is depleted,
   wherein the first temperature is changed to a second temperature of 26° C. at the time the feeding of step (b) is initiated; and
   (c) obtaining the recombinant protein.
7. A method for producing a recombinant protein in Pichia pastoris cells, comprising the steps of:
   (a) culturing yeast cells genetically modified to express the recombinant protein in a suitable cell culture medium with a molar ratio of potassium ions to phosphate ions is 1.5:1 to 2.8:1 or 1.6:1 to 2.7:1 and a molar ratio of potassium ions to sulfate ions is 2:1 to 4:1 or 2.3:1 to 4:1 and comprising a first carbon source at a first temperature of 28° C. and 37° C. and a first pH between 4.0 and 6.0, thereby obtaining a first culture;
   (b) feeding a second carbon source to said first culture when the first carbon source is depleted,
   wherein the first temperature is changed to a second temperature of 23° C. and 30° C. at the time the feeding of step (b) is initiated, wherein the second temperature is lower than the first temperature and wherein the first pH is changed to a second pH of 5.0 to 7.0 at the time the feeding of step (b) is initiated, wherein the second pH is higher than the first pH; and
   (c) obtaining the recombinant protein.
8. A method for producing a recombinant protein in Pichia pastoris cells, comprising the steps of:
   (a) culturing yeast cells genetically modified to express the recombinant protein in a suitable cell culture medium with a molar ratio of potassium ions to phosphate ions is 2.3:1 and a molar ratio of potassium ions to sulfate ions is 3.2:1 and comprising a first carbon source at a first temperature of 30° C. and a first pH of 5.0, thereby obtaining a first culture;
   (b) feeding a second carbon source to said first culture when the first carbon source is depleted,
   wherein the first temperature is changed to a second temperature of 26° C. at the time the feeding of step (b) is initiated and the first pH is changed to a second pH of 6.0 at the time the feeding of step (b) is initiated; and
   (c) obtaining the recombinant protein.
9. A method for producing a recombinant protein in Pichia pastoris cells, comprising the steps of:
   (a) culturing yeast cells genetically modified to express the recombinant protein in a suitable cell culture medium comprising glucose at a first temperature of 28° C. and 37° C., thereby obtaining a first culture;
   (b) feeding glucose to said first culture when the glucose in the cell culture medium is depleted,
   wherein the first temperature is changed to a second temperature of 23° C. and 30° C. at the time the feeding of step (b) is initiated, wherein the second temperature is lower than the first temperature; and
   (c) obtaining the recombinant protein.
10. A method for producing a recombinant protein in Pichia pastoris cells, comprising the steps of:
   (a) culturing yeast cells genetically modified to express the recombinant protein in a suitable cell culture medium comprising glucose at a first temperature of 30° C., thereby obtaining a first culture;
   (b) feeding glucose to said first culture when the glucose in the cell culture medium is depleted,
   wherein the first temperature is changed to a second temperature of 26° C. at the time the feeding of step (b) is initiated; and
   (c) obtaining the recombinant protein.
11. A method for producing a recombinant protein in Pichia pastoris cells, comprising the steps of:
   (a) culturing yeast cells genetically modified to express the recombinant protein in a suitable cell culture medium comprising glucose at a first temperature of 28° C. and 37° C. and a first pH between 4.0 and 6.0, thereby obtaining a first culture;
   (b) feeding glucose to said first culture when the glucose in the cell culture medium is depleted,
   wherein the first temperature is changed to a second temperature of 23° C. and 30° C. at the time the feeding of step (b) is initiated, wherein the second temperature is lower than the first temperature and wherein the first pH is changed to a second pH of 5.0 to 7.0 at the time the feeding of step (b) is initiated, wherein the second pH is higher than the first pH; and
   (c) obtaining the recombinant protein.
12. A method for producing a recombinant protein in Pichia pastoris cells, comprising the steps of:
   (a) culturing yeast cells genetically modified to express the recombinant protein in a suitable cell culture medium comprising glucose at a first temperature of 30° C. and a first pH of 5.0, thereby obtaining a first culture;
   (b) feeding glucose to said first culture when the glucose in the cell culture medium is depleted,
   wherein the first temperature is changed to a second temperature of 26° C. at the time the feeding of step (b) is initiated and the first pH is changed to a second pH of 6.0 at the time the feeding of step (b) is initiated; and
   (c) obtaining the recombinant protein.
13. A method for producing a recombinant protein in Pichia pastoris cells, comprising the steps of:
   (a) culturing yeast cells genetically modified to express the recombinant protein in a suitable cell culture medium with a molar ratio of potassium ions to phosphate ions is 1.5:1 to 2.8:1 or 1.6:1 to 2.7:1 and a molar ratio of potassium ions to sulfate ions is 2:1 to 4:1 or 2.3:1 to 4:1 and comprising glucose at a first temperature of 28° C. and 37° C., thereby obtaining a first culture;
   (b) feeding glucose to said first culture when the glucose in the cell culture medium is depleted,
   wherein the first temperature is changed to a second temperature of 23° C. and 30° C. at the time the feeding of step (b) is initiated, wherein the second temperature is lower than the first temperature; and
   (c) obtaining the recombinant protein.
14. A method for producing a recombinant protein in Pichia pastoris cells, comprising the steps of:
   (a) culturing yeast cells genetically modified to express the recombinant protein in a suitable cell culture medium with a molar ratio of potassium ions to phosphate ions is 2.3:1 and a molar ratio of potassium ions to sulfate ions is 3.2:1 and comprising glucose at a first temperature of 30° C., thereby obtaining a first culture;
   (b) feeding glucose to said first culture when the glucose in the cell culture medium is depleted,
   wherein the first temperature is changed to a second temperature of 26° C. at the time the feeding of step (b) is initiated; and
   (c) obtaining the recombinant protein.
15. A method for producing a recombinant protein in Pichia pastoris cells, comprising the steps of:
   (a) culturing yeast cells genetically modified to express the recombinant protein in a suitable cell culture medium with a molar ratio of potassium ions to phosphate ions is 1.5:1 to 2.8:1 or 1.6:1 to 2.7:1 and a molar ratio of potassium ions to sulfate ions is 2:1 to 4:1 or 2.3:1 to 4:1 and comprising glucose at a first temperature of 28° C. and 37° C. and a first pH between 4.0 and 6.0, thereby obtaining a first culture;
   (b) feeding glucose to said first culture when the glucose in the cell culture medium is depleted,
   wherein the first temperature is changed to a second temperature of 23° C. and 30° C. at the time the feeding of step (b) is initiated, wherein the second temperature is lower than the first temperature and wherein the first pH is changed to a second pH of 5.0 to 7.0 at the time the feeding of step (b) is initiated, wherein the second pH is higher than the first pH; and
   (c) obtaining the recombinant protein.
16. A method for producing a recombinant protein in Pichia pastoris cells, comprising the steps of:
   (a) culturing yeast cells genetically modified to express the recombinant protein in a suitable cell culture medium with a molar ratio of potassium ions to phosphate ions is 2.3:1 and a molar ratio of potassium ions to sulfate ions is 3.2:1 and comprising glucose at a first temperature of 30° C. and a first pH of 5.0, thereby obtaining a first culture;
   (b) feeding glucose to said first culture when the glucose in the cell culture medium is depleted,
   wherein the first temperature is changed to a second temperature of 26° C. at the time the feeding of step (b) is initiated and the first pH is changed to a second pH of 6.0 at the time the feeding of step (b) is initiated; and
   (c) obtaining the recombinant protein.
17. A method for producing a recombinant lipase in Pichia pastoris cells, comprising the steps of:
   (a) culturing yeast cells genetically modified to express the lipase in a suitable cell culture medium comprising a first carbon source at a first temperature of 28° C. and 37° C., thereby obtaining a first culture;
   (b) feeding a second carbon source to said first culture when the first carbon source is depleted,
   wherein the first temperature is changed to a second temperature of 23° C. and 30° C. at the time the feeding of step (b) is initiated, wherein the second temperature is lower than the first temperature; and
   (c) obtaining the recombinant lipase.
18. A method for producing a lipase in Pichia pastoris cells, comprising the steps of:
   (a) culturing yeast cells genetically modified to express the lipase in a suitable cell culture medium comprising a first carbon source at a first temperature of 30° C., thereby obtaining a first culture;
   (b) feeding a second carbon source to said first culture when the first carbon source is depleted,
   wherein the first temperature is changed to a second temperature of 26° C. at the time the feeding of step (b) is initiated; and
   (c) obtaining the recombinant lipase.
19. A method for producing a recombinant lipase in Pichia pastoris cells, comprising the steps of:
   (a) culturing yeast cells genetically modified to express the lipase in a suitable cell culture medium comprising a first carbon source at a first temperature of 28° C. and 37° C. and a first pH between 4.0 and 6.0, thereby obtaining a first culture;
   (b) feeding a second carbon source to said first culture when the first carbon source is depleted,
   wherein the first temperature is changed to a second temperature of 23° C. and 30° C. at the time the feeding of step (b) is initiated, wherein the second temperature is lower than the first temperature and wherein the first pH is changed to a second pH of 5.0 to 7.0 at the time the feeding of step (b) is initiated, wherein the second pH is higher than the first pH; and
   (c) obtaining the recombinant lipase.
20. A method for producing a recombinant lipase in Pichia pastoris cells, comprising the steps of:
   (a) culturing yeast cells genetically modified to express the lipase in a suitable cell culture medium comprising a first carbon source at a first temperature of 30° C. and a first pH of 5.0, thereby obtaining a first culture;
   (b) feeding a second carbon source to said first culture when the first carbon source is depleted,
   wherein the first temperature is changed to a second temperature of 26° C. at the time the feeding of step (b) is initiated and the first pH is changed to a second pH of 6.0 at the time the feeding of step (b) is initiated; and
   (c) obtaining the recombinant lipase.
21. A method for producing a recombinant lipase in Pichia pastoris cells, comprising the steps of:
   (a) culturing yeast cells genetically modified to express the recombinant lipase in a suitable cell culture medium with a molar ratio of potassium ions to phosphate ions is 1.5:1 to 2.8:1 or 1.6:1 to 2.7:1 and a molar ratio of potassium ions to sulfate ions is 2:1 to 4:1 or 2.3:1 to 4:1 and comprising a first carbon source at a first temperature of 28° C. and 37° C., thereby obtaining a first culture;
   (b) feeding a second carbon source to said first culture when the first carbon source is depleted,
   wherein the first temperature is changed to a second temperature of 23° C. and 30° C. at the time the feeding of step (b) is initiated, wherein the second temperature is lower than the first temperature; and
   (c) obtaining the recombinant lipase.
22. A method for producing a recombinant lipase in Pichia pastoris cells, comprising the steps of:
   (a) culturing yeast cells genetically modified to express the recombinant lipase in a suitable cell culture medium with a molar ratio of potassium ions to phosphate ions is 2.3:1 and a molar ratio of potassium ions to sulfate ions is 3.2:1 and comprising a first carbon source at a first temperature of 30° C., thereby obtaining a first culture;
   (b) feeding a second carbon source to said first culture when the first carbon source is depleted,
   wherein the first temperature is changed to a second temperature of 26° C. at the time the feeding of step (b) is initiated; and
   (c) obtaining the recombinant lipase.
23. A method for producing a recombinant lipase in Pichia pastoris cells, comprising the steps of:
   (a) culturing yeast cells genetically modified to express the recombinant lipase in a suitable cell culture medium with a molar ratio of potassium ions to phosphate ions is 1.5:1 to 2.8:1 or 1.6:1 to 2.7:1 and a molar ratio of potassium ions to sulfate ions is 2:1 to 4:1 or 2.3:1 to 4:1 and comprising a first carbon source at a first temperature of 28° C. and 37° C. and a first pH between 4.0 and 6.0, thereby obtaining a first culture;
   (b) feeding a second carbon source to said first culture when the first carbon source is depleted,
   wherein the first temperature is changed to a second temperature of 23° C. and 30° C. at the time the feeding of step (b) is initiated, wherein the second temperature is lower than the first temperature and wherein the first pH is changed to a second pH of 5.0 to 7.0 at the time the feeding of step (b) is initiated, wherein the second pH is higher than the first pH; and
   (c) obtaining the recombinant lipase.
24. A method for producing a recombinant lipase in Pichia pastoris cells, comprising the steps of:
   (a) culturing yeast cells genetically modified to express the recombinant lipase in a suitable cell culture medium with a molar ratio of potassium ions to phosphate ions is 2.3:1 and a molar ratio of potassium ions to sulfate ions is 3.2:1 and comprising a first carbon source at a first temperature of 30° C. and a first pH of 5.0, thereby obtaining a first culture;
   (b) feeding a second carbon source to said first culture when the first carbon source is depleted,
   wherein the first temperature is changed to a second temperature of 26° C. at the time the feeding of step (b) is initiated and the first pH is changed to a second pH of 6.0 at the time the feeding of step (b) is initiated; and
   (c) obtaining the recombinant lipase.
25. A method for producing a recombinant lipase in Pichia pastoris cells, comprising the steps of:
   (a) culturing yeast cells genetically modified to express the recombinant lipase in a suitable cell culture medium comprising glucose at a first temperature of 28° C. and 37° C., thereby obtaining a first culture;
   (b) feeding glucose to said first culture when the glucose in the cell culture medium is depleted,
   wherein the first temperature is changed to a second temperature of 23° C. and 30° C. at the time the feeding of step (b) is initiated, wherein the second temperature is lower than the first temperature; and
   (c) obtaining the recombinant lipase.
26. A method for producing a recombinant lipase in Pichia pastoris cells, comprising the steps of:
   (a) culturing yeast cells genetically modified to express the recombinant lipase in a suitable cell culture medium comprising glucose at a first temperature of 30° C., thereby obtaining a first culture;
   (b) feeding glucose to said first culture when the glucose in the cell culture medium is depleted,
   wherein the first temperature is changed to a second temperature of 26° C. at the time the feeding of step (b) is initiated; and
   (c) obtaining the recombinant lipase.
27. A method for producing a recombinant lipase in Pichia pastoris cells, comprising the steps of:
   (a) culturing yeast cells genetically modified to express the recombinant lipase in a suitable cell culture medium comprising glucose at a first temperature of 28° C. and 37° C. and a first pH between 4.0 and 6.0, thereby obtaining a first culture;
   (b) feeding glucose to said first culture when the glucose in the cell culture medium is depleted,
   wherein the first temperature is changed to a second temperature of 23° C. and 30° C. at the time the feeding of step (b) is initiated, wherein the second temperature is lower than the first temperature and wherein the first pH is changed to a second pH of 5.0 to 7.0 at the time the feeding of step (b) is initiated, wherein the second pH is higher than the first pH; and
   (c) obtaining the recombinant lipase
28. A method for producing a recombinant lipase in Pichia pastoris cells, comprising the steps of:
   (a) culturing yeast cells genetically modified to express the recombinant lipase in a suitable cell culture medium comprising glucose at a first temperature of 30° C. and a first pH of 5.0, thereby obtaining a first culture;
   (b) feeding glucose to said first culture when the glucose in the cell culture medium is depleted,
   wherein the first temperature is changed to a second temperature of 26° C. at the time the feeding of step (b) is initiated and the first pH is changed to a second pH of 6.0 at the time the feeding of step (b) is initiated; and
   (c) obtaining the recombinant lipase
29. A method for producing a recombinant lipase in Pichia pastoris cells, comprising the steps of:
   (a) culturing yeast cells genetically modified to express the recombinant lipase in a suitable cell culture medium with a molar ratio of potassium ions to phosphate ions is 1.5:1 to 2.8:1 or 1.6:1 to 2.7:1 and a molar ratio of potassium ions to sulfate ions is 2:1 to 4:1 or 2.3:1 to 4:1 and comprising glucose at a first temperature of 28° C. and 37° C., thereby obtaining a first culture;
   (b) feeding glucose to said first culture when the glucose in the cell culture medium is depleted,
   wherein the first temperature is changed to a second temperature of 23° C. and 30° C. at the time the feeding of step (b) is initiated, wherein the second temperature is lower than the first temperature; and
   (c) obtaining the recombinant lipase.
30. A method for producing a recombinant lipase in Pichia pastoris cells, comprising the steps of:
   (a) culturing yeast cells genetically modified to express the recombinant lipase in a suitable cell culture medium with a molar ratio of potassium ions to phosphate ions is 2.3:1 and a molar ratio of potassium ions to sulfate ions is 3.2:1 and comprising glucose at a first temperature of 30° C., thereby obtaining a first culture;
   (b) feeding glucose to said first culture when the glucose in the cell culture medium is depleted,
   wherein the first temperature is changed to a second temperature of 26° C. at the time the feeding of step (b) is initiated; and
   (c) obtaining the recombinant lipase.
31. A method for producing a recombinant lipase in Pichia pastoris cells, comprising the steps of:
   (a) culturing yeast cells genetically modified to express the recombinant lipase in a suitable cell culture medium with a molar ratio of potassium ions to phosphate ions is 1.5:1 to 2.8:1 or 1.6:1 to 2.7:1 and a molar ratio of potassium ions to sulfate ions is 2:1 to 4:1 or 2.3:1 to 4:1 and comprising glucose at a first temperature of 28° C. and 37° C. and a first pH between 4.0 and 6.0, thereby obtaining a first culture;
   (b) feeding glucose to said first culture when the glucose in the cell culture medium is depleted,
   wherein the first temperature is changed to a second temperature of 23° C. and 30° C. at the time the feeding of step (b) is initiated, wherein the second temperature is lower than the first temperature and wherein the first pH is changed to a second pH of 5.0 to 7.0 at the time the feeding of step (b) is initiated, wherein the second pH is higher than the first pH; and
   (c) obtaining the recombinant lipase.
32. A method for producing a recombinant lipase in Pichia pastoris cells, comprising the steps of:
   (a) culturing yeast cells genetically modified to express the recombinant lipase in a suitable cell culture medium with a molar ratio of potassium ions to phosphate ions is 2.3:1 and a molar ratio of potassium ions to sulfate ions is 3.2:1 and comprising glucose at a first temperature of 30° C. and a first pH of 5.0, thereby obtaining a first culture;
   (b) feeding glucose to said first culture when the glucose in the cell culture medium is depleted,
   wherein the first temperature is changed to a second temperature of 26° C. at the time the feeding of step (b) is initiated and the first pH is changed to a second pH of 6.0 at the time the feeding of step (b) is initiated; and
   (c) obtaining the recombinant lipase.

Any of the specific embodiments 1-32 can be combined with any embodiments disclosed herein.

The invention is further illustrated in the following examples which are not intended to be in any way limiting to the scope of the invention as claimed. The numerous possible variations that are obvious to a person skilled in the art also fall within the scope of the invention.

### EXAMPLES

Unless otherwise stated the following experiments were performed using standard equipment, methods, chemicals, and biochemicals as used in genetic engineering and fermentative production of chemical compounds, in particular proteins, by cultivation of yeast cells. See also Sambrook et al. (Molecular Cloning: A Laboratory Manual. 2nd edition, Cold Spring Harbor Laboratory, Cold Spring Harbor Laboratory Press, Cold Spring Harbor, N.Y., 1989) and Chmiel et al. (Bioprozesstechnik 1. Einführung in die Bioverfahrenstechnik, Gustav Fischer Verlag, Stuttgart, 1991).

### Example 1: Pichia pastoris Strain Used in the Experiments

Gene parts were cloned into the pPICz or pAO815 expression vectors (Invitrogen). Expression vectors were linearized by restriction digest and the vector backbone was isolated using gel electrophoresis and purification. New DNA parts such as promoters, nucleic acid sequences encoding signal peptides, the recombinant protein, or the selection markers were cloned into the vector backbones by seamless ligation using the GeneArt Seamless cloning and assembly kit (Invitrogen) or by restriction ligation using T4 ligase (ThermoFischer).

The cloned constructs were transformed into an E. coli cloning host such as One Shot TOP10 (Invitrogen) or XL1-blue (Agilent), sequence verified, and then plasmid was purified. Purified expression vector plasmids were linearized by restriction digest and transformed into K. phaffi by electroporation. Transformants screened for transformation by zeomycin selection or by selection on minimal media. Individual colonies were screened first in microtiter plates for expression before growth in fermenters.

Details of the strains are disclosed in international patent application PCT/US19/61929 filed on Nov. 18, 2019.

### Example 2: Protein Quantitation

Lipase protein titers in the whole broth were measured by lipase activity by incubating the whole broth with p-octanoate as substrate in 50 mM HEPES pH 7.5 at a temperature of 26° C. for 10 minutes. Whole broth specific lipase activity was compared to a lipase gold standard which had been quantitated by amino acid analysis (AAA).

### Example 3: Effect of Temperature Shift on Protein Titer

A seed culture of the Pichia pastoris strain as described in Example 1 was inoculated into cell culture medium at a concentration of 10% v/v. The fermentation media formulation contained ammonium phosphate (10 g/L), calcium sulfate dihydrate (1.1 g/L), potassium phosphate monobasic (15.6 g/L), potassium phosphate dibasic (0.7 g/L), potassium sulfate (3.4 g/L), ammonium sulfate (11.8 g/L), magnesium sulfate hepta hydrate (8.2 g/L), copper sulfate penta hydrate (99 mg/L), iron sulfate hepta hydrate (1 g/L), zinc sulfate hepta hydrate (330 mg/L), manganese sulfate monohydrate (50 mg/L), potassium iodide (1.5 mg/L), antifoam, vitamins and dextrose corn syrup (to 30 g/L dextrose).

The fermentation was controlled at a dissolved oxygen level of 20% of saturation via PID control cascade to agitation, a pH of 5.0 via PID control cascade to a concentrated ammonium hydroxide solution and a temperature of 30.0 C via PID control cascade to jacket heating/cooling.

Upon depletion of the batched initially batched dextrose, a concentrated dextrose feed solution was added dose-wise, with 1 g dextrose/L being added with each dose. The criteria for each dextrose dose addition is depletion of the prior dose. This feed strategy continued for the remainder of the fermentation. Upon reaching an oxygen uptake rate of 150 mmol/L/hr, the temperature was ramped linearly from 30.0° C. to 28.0° C. over one hour, after which the new value was held constant for the remainder of the fermentation.

In another experiment, the temperature was ramped linearly from 30.0° C. to 26.0° C. instead of from 30.0° C. to 28.0° C.

The culture was stopped after 120 hours and the lipase titer was determined as described in Example 2.

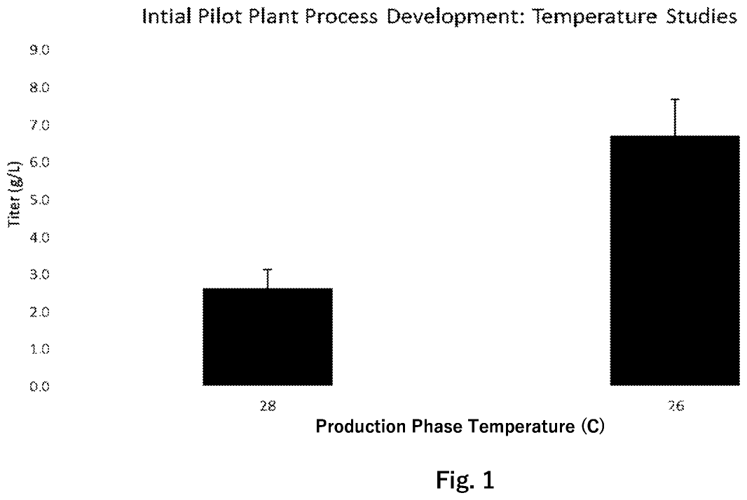

FIG. 1 shows that the temperature shift to 26.0° C. instead of to 28.0° C. increased the product titer by more than two-fold.

### Example 4: Effect of Media Composition on Protein Titer

In other experiments, the culturing was described as above in example 3, but in a medium containing 0.3 g/L calcium sulfate dihydrate, 2 g/L potassium phosphate monobasic, 10 g/L potassium phosphate dibasic, 3.2 g/L potassium sulfate, 1.6 g/L ammonium sulfate, 3.5 g/L magnesium sulfate hepta hydrate, 99 mg/L copper sulfate penta hydrate, 1 g/L iron sulfate hepta hydrate, 330 mg/L zinc sulfate hepta hydrate, 50 mg/L manganese sulfate monohydrate, 1.5 mg/L potassium iodide, antifoam, vitamins and dextrose corn syrup to 15 g/L dextrose, i.e. in a medium in which the ratio of phosphate and sulfate to potassium were lower than in the medium used in Example 3.

In these experiments, upon completion of the initially batched dextrose, a concentrated dextrose feed solution was added continuously at a rate of 3.5 g dextrose/L/hr. The dextrose solution feed rate was then increased exponentially at a specific rate of 0.15 hr−1 until reaching a maximum of 18.8 g dextrose/L/hr. The dextrose solution feed rate was then ramped down linearly from 18.8 g dextrose/L/hr to 6.5 g dextrose/L/hr over 14 hours. Finally, the dextrose feed rate was set to deliver 3.8 g dextrose/L/hr for the remainder of the fermentation.

When the maximum feed rate was reached, the temperature was ramped down linearly from 30.0° C. to 26.0° C. over one hour, after which the temperature was kept constant for the remainder of the fermentation.

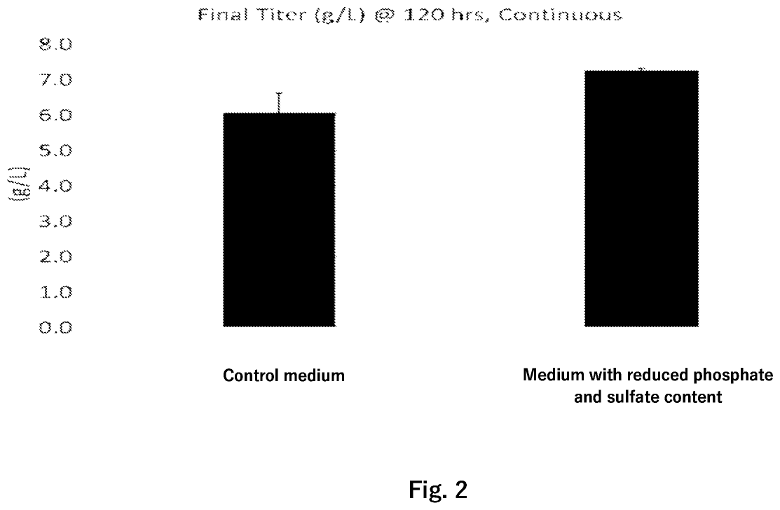

FIG. 2 shows that the use of a medium with a reduced concentration of phosphate and sulfate relative to the concentration of potassium increases the titer of the recombinant protein when using the same feeding strategy described in this example.

### Example 5: Effect of Feeding Strategy on the Scalability

The culturing was performed as described in Example 4 above in two different scales. In the R&D scale the fermenter size was 1 liter and in the pilot scale the fermenter size was 40 liters. In the pilot scale the fermentors were run with a back pressure of 10 psi.

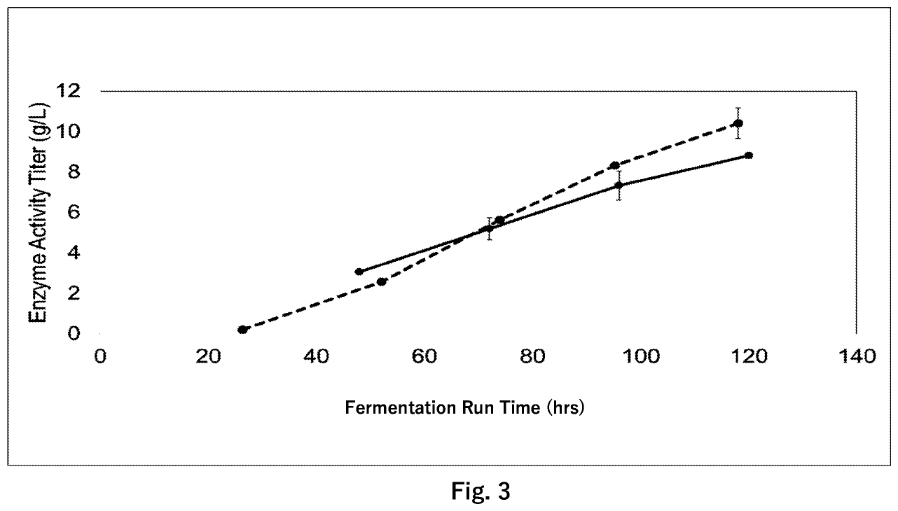

FIG. 3 shows that the continuous feed provided scalability of the process from R&D scale to pilot scale.

### Example 6: Effect of Temperature Shift Timing on Protein Titer

In another experiment, the effect of the timing of the temperature shift on product titer was investigated. The culturing was performed as described in example 4 above, but in addition to performing the temperature shift when the feed rate was maximal (“normal”), the temperature shift was performed either at the same time when the feeding was initiated (“early”) or at the time the feed rate has returned from a maximum feed rate to a constant final feed rate (“late”).

Table 1 shows that the timing of the temperature shift strongly influences the product titer and that an early temperature shift provides the highest protein titer:

| Temperature shift timing | Product titer |
|---|---|
| early | approx. 11.0 g/l |
| normal | approx. 8.5 g/l |
| late | approx. 2.0 g/l |

1. Method for producing a recombinant protein in yeast cells, comprising the steps of:
(a) culturing yeast cells genetically modified to express the recombinant protein in a suitable cell culture medium comprising a first carbon source at a first temperature, thereby obtaining a first culture;
(b) feeding a second carbon source to said first culture when the first carbon source is depleted, wherein the first temperature is changed to a second temperature at the time the feeding of step (b) is initiated; and
(c) obtaining the recombinant protein.

2. Method according to claim 1, wherein the second temperature is lower than the first temperature, and wherein the second temperature is 1 to 8° C. lower than the first temperature.

3. (canceled)

4. Method according to claim 2, wherein the first temperature is 30° C. and the second temperature is 26° C.

5. (canceled)

6. Method according to claim 2, wherein the first temperature is changed to the second temperature linearly over a period of time.

7. Method according to claim 2, wherein the first temperature is changed to the second temperature linearly over a period of 30 minutes to two hours.

8. Method according to claim 1, wherein the culture pH is changed from a first pH to a second pH at the time the feeding of step (b) is initiated.

9. Method according to claim 8, wherein the second pH is higher than the first pH.

10. Method according to claim 8, wherein the second pH is 0.5 to 2 pH units higher than the first pH.

11. Method according to claim 8, wherein the first pH is 5.0 and the second pH is 6.0.

12. (canceled)

13. Method according to claim 8, wherein the first pH is changed to the second pH linearly over a period of time.

14. Method according to claim 8, wherein the first pH is changed to the second pH linearly over a period of 30 minutes to two hours.

15. Method according IQ claim 1, wherein the first and the second carbon source are the same.

16. Method according to claim 1, wherein the first and/or the second carbon source is glucose.

17. Method according to claim 1, wherein the culture medium is a defined medium.

18. Method according to claim 1, wherein the concentration of potassium ions in the culture medium is higher than the concentration of sulfate ions and/or phosphate ions.

19. Method according to claim 18, wherein the molar ratio of potassium ions to phosphate ions is 1.5:1 to 2.8:1, and the molar ratio of potassium ions to sulfate ions is 2:1 to 4:1.

20. (canceled)

21. Method according to claim 1, wherein the ammonium concentration in the culture medium is 5 mM to 80 mM, the calcium concentration in the culture medium is 0.5 mM to 3 mM, the magnesium concentration in the culture medium is 5 mM to 30 mM.

22. (canceled)

23. (canceled)

24. Method according to claim 1, wherein the feeding of the second carbon source is continuous.

25. Method according to claim 24, wherein the feed rate is increased exponentially from an initial feed rate to a maximum feed rate.

26. Method according to claim 25, wherein the feed rate is decreased linearly from the maximum feed rate to an intermediate feed rate and then decreased to a final feed rate.

27. Method for producing a recombinant protein in yeast cells, comprising the steps of:
(a) culturing yeast cells genetically modified to express the recombinant protein in a suitable cell culture medium comprising a first carbon source, potassium ions, sulfate ions and phosphate ions at a first temperature, wherein the concentration of the potassium ions in the culture medium is higher than the concentration of the sulfate ions and/or of the phosphate ions; and
(b) obtaining the recombinant protein.

28. Method according to claim 27, wherein the molar ratio of potassium ions to phosphate ions is 1.5:1 to 2.8:1, the molar ratio of potassium ions to sulfate ions is 2:1 to 4:1.

29. (canceled)

30. Method according to claim 27, wherein the ammonium concentration in the culture medium is 5 mM to 80 mM, wherein the calcium concentration in the culture medium is 0.5 mM to 3 mM, and wherein the calcium concentration in the culture medium is 0.5 mM to 3 mM.

31. (canceled)

32. (canceled)

33. Method according to claim 27, wherein the culture medium is a defined medium.

34. Method according to claim 27, further comprising a step (a′) of feeding a second carbon source to the yeast cell culture of (a) when the first carbon source is depleted.

35. Method according to claim 34, wherein the first temperature is changed to a second temperature at the time the feeding of step (a′) is initiated.

36. Method according to claim 34, wherein the second temperature is lower than the first temperature, and wherein the second temperature is 1 to 5° C. lower than the first temperature.

37. (canceled)

38. Method according to claim 36 27 to 37, wherein the first temperature is 30° C. and the second temperature is 26° C.

39. (canceled)

40. Method according to claim 34, wherein the first temperature is changed to the second temperature linearly over a period of time.

41. (canceled)

42. Method according to claim 34 to 41, wherein the culture pH is changed from a first pH to a second pH at the time the feeding of step (a′) is initiated.

43. Method according to claim 42, wherein the second pH is higher than the first pH.

44. Method according to claim 42, wherein the second pH is 0.5 to 2 pH units higher than the first pH.

45. Method according to claim 42, wherein the first pH is 5.0, and the second pH is 6.0.

46. (canceled)

47. Method according to claim 42, wherein the first pH is changed to the second pH linearly over a period of time.

48. (canceled)

49. Method according to claim 34, wherein the first and the second carbon source are the same.

50. Method according to claim 34, wherein the first and/or the second carbon source is glucose.

51. Method according to claim 34, wherein the feeding of the second carbon source is continuous.

52. Method according to claim 51, wherein the feed rate is increased exponentially from an initial feed rate to a maximum feed rate.

53. Method according to claim 52, wherein the feed rate is decreased linearly from the maximum feed rate to an intermediate feed rate and then decreased to a final feed rate.

54. Method according to claim 1, wherein the yeast is a methylotropic yeast.

55. Method according to claim 54, wherein the yeast is Pichia pastoris.

56. Method according to claim 54, wherein the recombinant protein is expressed under the control of a promoter which is not inducible by methanol.

57. Method according to claim 54, wherein the recombinant protein is expressed under the control of a promoter which is inducible by methanol.

58. Method according to claim 54, wherein the recombinant protein is an enzyme, a peptide, an antibody or antigen-binding fragment thereof, a protein antibiotic, a fusion protein, a vaccine or a vaccine-like protein or particle, a growth factor, a hormone or a cytokine, and wherein the recombinant protein is a lipase, protease, alpha-amylase, beta-amylase, glucoamylase, xylanase, mannanase, glucanase, cellulase, or phytase.

59. (canceled)


SEQUENCE LISTING (12 sequences)

SEQ ID NO: 1 - protein, 317 aa, artificial
  Lipase 062
AITASQLDYENFKFYIQHGAAAYCNSETASGQKITCSDNGCKGVEANNAIIVASFVGKGTGIGGYVSTDNVRKEIVLSIRGSSNIRNWLTNVDFGQSSCSYVRDCGVHTGFRNAWDEIAQRARDAVAKARTMNPSYKVIATGHSLGGAVATLGAADLRSKGTAVDIFTFGAPRVGNAELSAFITAQAGGEFRVTHGRDPVPRLPPIVFGYRHTSPEYWLAGGASTKTDYTVNDIKVCEGAANLACNGGTLGLDIIAHLRYFQDTDACTAGGISWKRGDKAKRDEIPKRQEGMTDEELEQKLNDYVAMDKEYVESNKM

SEQ ID NO: 2 - DNA, 954 bp, artificial
  Lipase 062 nucleic acid sequence
gccattactgcttctcaattggactacgaaaacttcaagttttacatccagcacggtgccgctgcttactgtaactccgaaactgcctctggtcaaaagatcacttgttccgacaacggttgcaaaggtgtcgaagctaacaacgctattattgtcgcctctttcgttggaaaaggtactggtattggtggttacgtttctactgataacgttagaaaggagatcgttttgtctattagaggttcttccaacattcgtaactggttgactaacgtcgacttcggacaatcctcttgttcttacgttagagattgtggagttcacactggtttcagaaatgcttgggacgagattgcccaaagagctagagacgctgtcgctaaagctagaactatgaacccatcttacaaggttatcgctactggtcactctttgggtggtgctgttgccactttgggtgctgctgatttgagatccaagggtactgccgtcgatatctttacttttggtgccccaagagttggtaacgctgagttgtccgctttcatcactgctcaggctggtggtgagttcagagttactcacggacgtgatccagttccacgtttgccacctatcgtcttcggttacagacacacctctccagagtactggttggctggtggtgcttccaccaagactgattatactgttaacgatatcaaggtttgtgaaggtgccgctaacttggcctgtaatggtggtactttgggattggatatcattgctcatttgagatacttccaagacactgacgcctgtactgctggtggtatctcctggaagagaggtgacaaagctaagagagatgagattccaaaaagacaagaaggaatgactgatgaggagttggaacaaaaactgaacgactatgtcgccatggataaggagtacgttgagtccaacaagatgtaa

SEQ ID NO: 3 - protein, 317 aa, artificial
  LIP120 amino acid sequence
AITASQLDYENFKFYIQHGAAAYCNSETASGQKITCSDNGCKGVEANNAIIVASFVGKGTGIGGYVSTDNVRKEIVLSIRGSHNLRNWLTNVDFGQSSCSYVRDCGVHTGFRNAWDEIAQRARDAVAKARTMNPSYKVIATGHSLGGAVATLGAADLRSKGTAVDIFTFGAPRVGNAELSAFITAQAGGEFRVTHGRDPVPRLPPIVFGYRHTSPEYWLAGGASTKTDYTVNDIKVCEGAANLACNGGTLGLDIIAHLRYFQDTDACGAGGISWKRGDKAKRDEIPKRQEGMTDEELEQKLNDYVAMDKEYVESNKM

SEQ ID NO: 4 - DNA, 954 bp, artificial
  LIP120 nucleic acid sequence
gccattactgcttctcaattggactacgaaaacttcaagttttacatccagcacggtgccgctgcttactgtaactccgaaactgcctctggtcaaaagatcacttgttccgacaacggttgcaaaggtgtcgaagctaacaacgctattattgtcgcctctttcgttggaaaaggtactggtattggtggttacgtttctactgataacgttagaaaggagatcgttttgtctattagaggttctcacaacttgcgtaactggttgactaacgtcgacttcggacaatcctcttgttcttacgttagagattgtggagttcacactggtttcagaaatgcttgggacgagattgcccaaagagctagagacgctgtcgctaaagctagaactatgaacccatcttacaaggttatcgctactggtcactctttgggtggtgctgttgccactttgggtgctgctgatttgagatccaagggtactgccgtcgatatctttacttttggtgccccaagagttggtaacgctgagttgtccgctttcatcactgctcaggctggtggtgagttcagagttactcacggacgtgatccagttccacgtttgccacctatcgtcttcggttacagacacacctctccagagtactggttggctggtggtgcttccaccaagactgattatactgttaacgatatcaaggtttgtgaaggtgccgctaacttggcctgtaatggtggtactttgggattggatatcattgctcatttgagatacttccaagacactgacgcctgtggtgctggtggtatctcctggaagagaggtgacaaagctaagagagatgagattccaaaaagacaagaaggaatgactgatgaggagttggaacaaaaactgaacgactatgtcgccatggataaggagtacgttgagtccaacaagatgtaa

SEQ ID NO: 5 - DNA, 940 bp, Komagataella phaffii
agatctaacatccaaagacgaaaggttgaatgaaacctttttgccatccgacatccacaggtccattctcacacataagtgccaaacgcaacaggaggggatacactagcagcagaccgttgcaaacgcaggacctccactcctcttctcctcaacacccacttttgccatcgaaaaaccagcccagttattgggcttgattggagctcgctcattccaattccttctattaggctactaacaccatgactttattagcctgtctatcctggcccccctggcgaggttcatgtttgtttatttccgaatgcaacaagctccgcattacacccgaacatcactccagatgagggctttctgagtgtggggtcaaatagtttcatgttccccaaatggcccaaaactgacagtttaaacgctgtcttggaacctaatatgacaaaagcgtgatctcatccaagatgaactaagtttggttcgttgaaatgctaacggccagttggtcaaaaagaaacttccaaaagtcggcataccgtttgtcttgtttggtattgattgacgaatgctcaaaaataatctcattaatgcttagcgcagtctctctatcgcttctgaaccccggtgcacctgtgccgaaacgcaaatggggaaacacccgctttttggatgattatgcattgtctccacattgtatgcttccaagattctggtgggaatactgctgatagcctaacgttcatgatcaaaatttaactgttctaacccctacttgacagcaatatataaacagaaggaagctgccctgtcttaaacctttttttttatcatcattattagcttactttcataattgcgactggttccaattgacaagcttttgattttaacgacttttaacgacaacttgagaagatcaaaaaacaactaattattcgaaacg

SEQ ID NO: 6 - DNA, 1500 bp, artificial
  promoter pSD003
tcacattcatagcatctctcgcctgcaatagcttccacgataggaatatctgtgaaagtgaacatgctatttcgatgatataagactttaagatctggcatgtttgtgttggaggttaccctggggtcaataaccctaattatctccttcactaaaaatgatgaagattcttcggattcgtttttgaacagagttaatgccatttcttcgtcaatagaaaaatcaatatctggtatctcatcttttacatattgaggatttagttttcttccctttggatagtacattatgatcaatgtattcctgtctttattgataaagtattggcattctgcttcttgtacacctttgaattgtttgtctggaagtgactgacatttttccacattgctaacggtttggcacgaattacatctaaataaaatgtcttctccggattcgtgtattaagtgatactccaatgataaatccccacctatcgaaccagaatcggcattggccacagtcacaggtaactttaggtcttgaaaaatccttctataggcttcattgacattgtcataagacttaagaccatcttctttggtcaagtcaaaagaataggcatctttcatgagaaactctcgtcctctcaacaaacctcccctaggtctcaactcatctctatatttgcgggaaatttggtacacgagaaggggtaaatctttatatgacgaacataagtcaccaactaagtttgtgatttcctcttcacaagttggcactaaacagtagtctctatccttggagtctttgaacttgaacaattcattgttgtcccatctcttagttctctcccataaatgcttggaagacaggctacttaattccatttccagcccaccagcctgatccattcttttcctaattacattttgaagctttttataggtacggagtcctaatggaagccagtgaactattcctgctgcaggctggtaaataaaccttgattgaaggagcatatcatgagtagtaaggtcctttacagaaaatagtttacttccttgaagagaagtagaataaaacctcatgttgggtctccatgaaaggttcaaaggcattgatcctttaggtacttcaggatgtttaagtcatcaaactgtccatcaaaggtagtatagtatttaccatctagatagtgatgtatgggtgtaacacaacatttaaatgttgtaaattaacattaggactgagtccggagatgctattgtcacctaaatctattagaaagcacttcagttatatcatcgatagaggtttgaagataaacctattgttgataaataaccccattacccgtttacgtagcaaggttcaaaaatttgcttagatcggagctaaaaattcgactgacttctttcgaaaatgtggattatgcaagcaacgttgctatcggaatagtatataaggtcgatctgccccattacaaattgtaaagcaacaaacatcctacgcaaa

SEQ ID NO: 7 - DNA, 1500 bp, artificial
  promoter pSD004
tcagtttcacggttatgtgagctgtctccgcgtgaggcagtaacctctgtgtcatggatacaggctggtacacatttggcagtaggaacacaatctggtttagttgaaatatgggacgccacgacgtccaaatgtacaagatcaatgactgggcattcggcccgaacctcagcgctgagttggaaccgtcatgttttgagttctggttcaagagatcgcagtatcttacatcgggatgtacgtgcagcagctcactatacaagtcgcattgttgaacaccgccaagaggtttgtggcttacgttggaacgtggatgaaaacaagctggccagtggttccaatgataaccgtatgatggtatgggatgcactgcgtgtagaacagccccttatgaaagttgaagagcatactgcggctgttaaggcgttggcatggtcacctcatcaacgtggaatactggcttcgggtggaggtactgctgacagacgtatcaaggtgtggaatactttaacaggatccaagctgcacgatgttgatactggatctcaagtttgtaatctcttgtggtctcgcaattctaatgaattggtaagtactcatggatattctcgaaaccaagtcgttatttggaaatatccgcaaatgaagcaactagcatctttgactggtcatacttatcgagtcctttacctttccatgtcacctgatggaactacagtcgtaacgggggctggagacgaaactttaagattttggaactgtttcgagaagtcacgacaaagcggaggaggatcaatattactagacgcttttagtcagcttcgttaaattaccaccaaatttggtgcaaaagggcccatatggtgctacaaccaaaggaactttctaattttgataatgatgtcatttctctcatcgggatgaaaatagaagtcgaaaggatttttgtcactatttcaagccccacctgcagctggcagcatttctattgtttatgcattgtcatttatgggaaaactaagaaagttcctctccacccggactccactggtaaatatgcgatatcggaatcatgaccaacccatattttgatcctaatcatttcggttctagtctccgatcggactccgtaaaactgcggagtgaactccaacggagaatactgcagccaatctcatatttcatttgttatttgtccctcaactgtctcgataaggtcatctgtgtttgactagatgttcgtcattggcatgtcaaacaaggctagaccttacaatcatctcttacgaatgtaagtgaatgtaactatattttccttgctactttaacgaggttaaccaacccccgcacatccccacaccaccgctcttgataagcatctccgaaaatgcatgacgcgacaacttcaagcatgttgtatttactgagttttcagcctcactatcgatacctctataaatagaggcactttcgtctcttctccctccccacaagaaacca

SEQ ID NO: 8 - DNA, 1500 bp, artificial
  promoter pSD007
gtcctttccaaatttttggttgaaggcatcgcttaaattatgagcaggatcggtggaaataagcaggtatttcttgttaggattgtgaagggcaagctggatagatatagaagaagatgtcgtggttttaccgacaccccccttacctccaacaaagatccacttcagcgattcgtggttcacaattgatcgcaaacttggctctgcctcaatatccatggttgatgtctagttgagtggcgtttgtggtctcttgatgagttcaaggcgaaagaatatgataggaaagcatggtttgaacttttcgcgaaagaaggaatactgttccgcgagaaactccccggtgccagaaccttccattgaggttaatcggtgggaggtgttcgaatgacaatgtcagacaaggcgaacacgtcttgtgacaccagctggactaagaagattcggtatgcaccgaagaagaaggccgtgtctcaattggcaactttgcaacaaactacggaggaaaagtctcacaagcttttaaccaagttgaatcacgacgacaacgataaagaaatcctcaaccatctaacacatgaagtacaaagtagaaatgtgatcttattggacaaactagaggagctcaacaaggaactgggctggattaaagaccgaaaatgaggaaccatgagcactgggcgtttccagaaaaactgcaaccaacgatgggaaaatgataccacactactatggtcaccccacattgtgaaatttcaaaccaaaaaagatcaaccccataattccccagagggttttcccaacaattttccaacggacttgataatgagtcagatcatttgagcatattcatcttaccccttattccgtgacaatttacctattccattcaaagcatacggtatcccgtgaccttctcatggagatcattctccaccgatacagcatatacacagatatacccaactaatatcaattggaccttgatatggtcgaccttgatggtcccgtccaaccttaaaacttagtttaatgctatactttcgccttgaaccaaatctgtctccccctcaatcatctctatgcaagaaggtcaacactgattacgtgagcaacagccagcaatcgttcgagtccccgccaaaaaaggcggagttactgctccttgtgaccacaccccctgagaccacgtccctaaacgatccttgtcggttccttcgtccaattggcaattgccacgcatacgtgaatcgttattgtttcgcctaccttgcgtcattcgttccagaatgttcgacatactcctctagaacataccgtcacaccaccatcttaagttatcttcacgtgaccatgacgtacattgtagttgactaccccattctcatcattccgatgcggccaaaaatctctatataaagaccgtatcccctaatattctcttcttgttaagacattaacttagttaattcaccaattactcacttataaacaaacaaa

SEQ ID NO: 9 - DNA, 1501 bp, artificial
  pSD001
tccagtgtagcactaaaatctaatatcttcggctttatacttttttgttcatccgaaagcttacgaacaattctttctcctgttttattgtggatatagacaatttcgtcagtttcttggagagaagagttatttccggttttggctggccctataaacgggttcttggatttggatctagtaataaaaatgtcactgtcattctcggagctgaactttgtgttgtacgaagatgggttgttccactgttttgccagctcttcattgatgattttcttagtgggtgttcttggaggttcacgttgcctataatcttgacgttcttcttcatcactatcgatgccatcaaaattaagcgtccttattgcaggcttttgtgatttcaactgcaatccttctatctcttcatcagagctttcgaactgaatactatcactcaaaactggcgacattgcacatttccgcaaaccatttcgggaatctatgctagctcttctagacgataaagaacgaccggaaccaatacggggttgtgcaggtgggaataaatatgttggtttggattcttgacgtgaagaaggtattctagtcgatgaagtggttgataaggatatggcgtcactgagttgttttcttttcctatgttgcggtgttgggtcaggagttaattgattcacctccataactctggaatttcttgaatgtggggttttcagatgggcatctttcttgacggggttgtgagtaacggaggaacctggtgtcttgggtgtgaacggtgtttgagcctgtacgcggttacttctgggcggagtactcggagtcatgagagccattgattagaaggtgaatgagggagtcaccactctaagcaaacaaaatgaggtcgaagcaaaaaataaagtaaagtagcacttctggcaggttagatcaaagagtgacgggagatttgaagatggctggtttttccttagtcttggaagaggtttgtgtgggtatcagcgaatattccccgattaggcaaattagttgcattgaaattaacacgacatggtgatttgtggtaacaaatatctattggtggttggtgtgtgggtgtaatagtggtcgtgtcatgatgatggtgttcaggtgttgtcatagatcggtcttcagtaagagaaggaagcttggtgacgatcacagctatgatgtaatagaaattgctaagcaattgtgaggtgtgatgtattttgcagagcaattgtgcggtacaacggggtgttattgtcttcacaaggcatttattgcgaatttcgtagttgaaagaatattttagcacagggtgcttgacccctattgttgctcgctaaaccatgattgctaaatgatgacatagcaatcactttactaagattgctataaggacacctttcttagtataaatggacactcttttcccctgctaaacttcttttatttttcacacttaaacagttacaaaacacaaacacaactagaa

SEQ ID NO: 10 - DNA, 1501 bp, artificial
  promoter pSD002
gtgctaaaatctgaggtttacaagctgtgatgttcccctaagatctcacaatcgaacaatcgcgaagccaatgcaagttgtttaaggggaaacgactcactattcctgaaattagtattcaaaacttggtccggaagaacaatgaggcggccgttaaaatactcacgtaaacggtgtctacaagcgcattaaaatccgtttgaattcaagcaaaagccaccagaggcttatgcttggttatacccagcattgacctttggtatgagcatctgaaaaacaaccaggtgttgcaaagttaaacatccttctttgttcatatagaacccactattcatggtactccccaatcgaatttcacattctggttttgaaattacacaccacgttagcttataagatttcatataacttattgatatacggtttccattgttcgaatagttgaggttgtatgtaattcgattgaaggggccatttttgtttcctacttttcctgggagcttatccgatgcgcttcaaagctggaattgtaaatatagagaaaaagaaggatgttgttttattcttgaaagagtataattttacttctagcaactctcccacttcgcttgacttcatttatttcttgggcacataggcgtagtaatctagaccaacagataatttgccggaatgatatagcgattggaaaatgaactgaaattttttgctgtctttcaatttgacgggcagttcatcagtgaccgaccatataaatacgttgagaatgttattcttcctcgtagttgaagtggcttcataatttcagaactcaatagataaactaggatgttttaaagcaattaatgctcacaagtaaggagcgactctcttgcttttcgaatactaaaagtatcgtcccaacccagaaaaaaagacctcttaactgcaaaataaactctatatatttcttctaaaacagtttcaggttggatagtatcgcattctcatcacttctaactagtaggccatgagatatattaacgtttacttgagttctaagttctccgaattagatgcacagcacaaacaagattaggtttcacttggtacaaaatacgaacagagtttaaggtcgtaatttcatttcgttattgatccccacaatctattcttatcacagtcatcagatagtcgcgaaaaagcatgcagaaaagggggtcgtccctatctaagttgtagcattacaacaaatatgactacactcagtgtcgcaatcggtatagccaacgctgcaaaatggattctactgagaatggtatgatgatcccaggatcaatttcccaaaaattaaaaaaagtaaaataaaaagcatcagatattagggaggtggtaagattgctctgcaagcgatcacgagattttaggttttcctttatgtactatataaagcgcagattggatgccgcttttccctcctgggctatgataatatagcgaacgaaatacacgccaaaataaa

SEQ ID NO: 11 - DNA, 1500 bp, artificial
  promoter pSD005
agaagtactgttatgaatcgatcgacgtgacatgttgttgatggttctgacttcttgatgtccgcgttttctgtctctcaatagtggtgttcgggggaagtatggttctaatacttaacaggtaagatggttgcaatgagcacctggtaaagcaacttgaatttcctgccctgtctccgttaagttatattcgactcaaggtccttgcttcctgtctgttctgtaaaacttccctttggtgtcttctatatcaactttaaaaacaaggtagtgtgtcgagcgatagtactgtgtctttttccctatgaaaaaaatcgcaccatccaagacttctcaccttcaacagcttcaacatcatgttcggtccttttagagctacgctggtcgatctaggaggtctgctatggaaacgtccttggagaatgtccaaaccacagaaatatagactccgcaaaagaatgcaacttgtagactccaatatcgacattatttaccagggactgactgaggagggtctgtcttgcaaagtgatagataacttgaaacaaaacttcccaaaggagcatgaagtgctccccaaaaacaagtataccgtgtttaacaagacagccaaaaactatagaaagggtgttcatttggttccaaaatggaccaagaagtctttgagagagaaccccgagttcttctaattgcacatttcttcctgttcatagattatcccacacatagttgctcacaaaaaaatcactataattttcctccaccggcagtatatcactaacacctttatctttattgtagattataatctgatctttatccttagatgtatctatcatcaaccccatgctcttgaaaagcttgagtcttaacactgtcgaatcgtagttttcttgtagatcattcgatatcactgctttttcttgctcttctaattcgttgagattctgggtcaaactagagattgaattctgaaggtgattcatgttcatctccagatctgttattgattttgctaatttaaatttttcgtgttcaagctcttcgatactctttagggtctgttgacggtcttctgtttccaataattgcttgttgaactctttaagttcgtctctctgtttactgatacgtgacaacaaatctagctggtgatcgagtttaagtttccgtttggagctcaacagagaaagattttcattaatttggttgatagtttgcacgtccggttcgatctgaaaattctctatagtcgacctgattaaggacacagtctcttgaagatcggacattggatttatggagaagggagatcaaagcggaaccagttgcactgtttacctttccagtcgagatacttatcccacagggccctcactttccaggcagaagtcacctaggaggcgcatccctccgtttgcttccctcgcgacaaactcccctgtaaaagaaaacttcactgaatcgtacacctaatcatacgacactaacacagatata

SEQ ID NO: 12 - DNA, 1500 bp, artificial
  promoter pSD008
gtttctcttggggagatacttttttcgcgtgctcctccgtgcggaacttccttctgagcttctacctctcagattagtctaatcgcatcaggaataagactgagaatgcttttaaggagaggcttgagattggctaattgcgttccgaagtactctttcaaaaggagttatacccctctcaactacgattctctaaagaattatcgtaggcatgctcaggcgcctcaaccccatcagtttgacgccactagatgggaccaacaaccagttactaatgagcaaggagtaatactcccatccgactcaattgcaaacattctgagacaaccaactctggtcatagaacggcaaatggaaatgatgaatatatttttaggatttgagcaggcgaaccgatatgttatcatggatcctacaggaagtattttgggttacatgctagaaagggatctgggcatcaccaaagctatattgagacagatctaccgtttgcatcgaccttttacagtggatgtaatggatactgcaggaaatgtattaatgacaatcaagaggccgtttagtttcatcaattcgcacatcaaagctatattaccccctttcaggaacagcgacccagacgaacatgtaattggagaatccgttcaaagctggcatccttggagacgaagatacaatctatttacagcacaaattggcgaaaaggacactgtctacgatcagttcgggtacattgacgcaccgtttctttcctttgagtttcctgtactttcagaatctaggcaaacgctaggtgctgtctctagaaacttcgtgggctttgcaagagagcttttcacagatacaggagtttacatcatccgtatggggcctgaatcttttgtagggctagaagggaactacgggaacaatgtggcccaacatgcccttacgctggaccaaagggctgtattattagccaatgccgtttcaattgactttgattacttttctaggcactcgtcacacagtggtggcttcattgggtttgaggaatagacagggtctcgtcaactcagctcctgccaccaaaccaatcattgatcaacgagcacacttttgtccacgtgagatcgctttcgcttgcagaaagagcaatgcatgaaaacggcaaacgcaaaacgagcaaaaaaacgagtaaataactacaatttcaccaccaacagggtcaaagagcttttgagacactataaaaggggccctttccccccaggttccttgaaatcctcattcaattatgttttttactcataatttgactcaattggcatcttcttctttgttcatatacagtaattgatatgacgcttagtcattattagtgttctcgactagcagtggcgaaaaaagggggagttattttctagaaccgaccgcaaactataaaagaaagctgcccctcatatacctttcgaattctttattttctgtgtttcttccctatttaacatctacacaaaa